(19) United States
(12) Patent Application Publication
(10) Pub. No.: US 2024/0282469 A1
(43) Pub. Date: Aug. 22, 2024

(54) MONITORING A PERSON USING MACHINE LEARNING

(71) Applicant: Milbotix Ltd, Oxon (GB)

(72) Inventors: Zeke Sebastian STEER, Oxon (GB); Prabha Thirthahalli VENKATESH, Oxon (GB); Bachir Houssem Eddine ZELMAT, Oxon (GB)

(21) Appl. No.: 18/442,841

(22) Filed: Feb. 15, 2024

(30) Foreign Application Priority Data
Feb. 16, 2023 (GB) 2302219.7

(51) Int. Cl. G16H 80/00; A61B 5/00; A61B 5/0531; A61B 5/11; G06N 5/022; G16H 20/70; G16H 30/40

(52) U.S. Cl. CPC G16H 80/00; A61B 5/0531; A61B 5/11; A61B 5/6807; A61B 5/7264; G06N 5/022; G16H 20/70; G16H 30/40; A61B 2562/0219; A61B 2562/06

(57) ABSTRACT

A method and system for monitoring a person, e.g. at their home, or in a residential care setting, or in hospital. The process involves obtaining time series sensor data, in particular from one or more sensors of a sock worn by the person. The sensor data is processed, e.g. to obtain a sequence of observations of the sensor data that is processed using a machine learning classifier, in implementations to predict a state of stress of the person.

### FIELD

This specification relates to systems for monitoring a person using machine learning.

### BACKGROUND

There is a need to improve the care of people with dementia. People with dementia can experience escalating agitation and distress, and it can be difficult for them to communicate this. A similar problem is experienced by, for example, people with autism spectrum disorders and some learning disabilities, and by some hospital patients. In some settings people spend time in their own room and are not immediately visible to care staff, who may therefore not be able to pick up on heightened distress or agitation until it becomes verbalised and has significantly decreased wellbeing.

There is a need to be able to detect early signs of distress or agitation, for example to alert carers. One approach to detecting stress would be to capture heart rate, blood pressure, and cortisol levels but this would typically necessitate stationary equipment and a clinical environment, limiting applicability and real-time stress detection. Physiological and motion data can be captured from a wrist or chest-worn device, but wearable technology is often complex, uncomfortable, and/or stigmatising. Also the detection of stress is difficult, and there can be a complex constellation of physical and physiological reactions involved. Acoustic monitoring is becoming increasingly common in care homes but by the time a person is vocalising, e.g. shouting, they are already experiencing significant distress and it can be too late for effective intervention.

### SUMMARY

In one aspect there is therefore described a method of monitoring a person, e.g. at their home, or in a residential care setting, or in hospital.

The process involves obtaining time series sensor data from one or more sensors, in particular from a sock worn by the person. The time series sensor data comprises physiological data from one or more physiological sensors characterizing a physiological state of the person, and/or motion data from one or more motion sensors characterizing motion of the person.

The time series sensor data is processed using a machine learning classifier, in particular a trained machine learning classifier, to generate classification data that predicts one of a plurality of categories.

In some implementations the process obtains a sequence or window of observations of the time series sensor data, e.g. raw or processed sensor values, such normalized sensor values.

The sequence of observations is processed using the (trained) machine learning classifier to generate classification data that predicts, for the sequence of observations, the one of the plurality of categories.

In some implementations the categories characterize different respective predicted states of stress of the person.

By obtaining data from one or more sensors on one or a pair of socks worn by the person physiological data and/or motion data can be collected in a way that is less invasive, and that helps avoid the social discomfort that can come with wearing obvious healthcare devices. The socks can be washable, and can have a wireless connection to the machine learning classifier.

The time series sensor data could be processed directly by the (trained) machine learning classifier. However in implementations a sequence of observations is obtained from the time series sensor data, and the sequence of observations is processed using the (trained) machine learning classifier. This has an advantage in that the sequence of observations comprises multiple observations, and may thus encode features of and/or relationships between multiple observations, which in turn can improve the accuracy of the classification.

The described techniques can also be used to make different classifications, in particular motion classifications. That is, in some implementations the categories characterize different respective predicted motion states of the person.

Thus in another aspect there is described a method of monitoring a person that involves obtaining time series sensor data from one or more sensors of the person, e.g. from one or more sensors of a sock worn by the person. The time series sensor data comprising a series of one or both of physiological data from one or more physiological sensors characterizing a physiological state of the person and motion data from one or more motion sensors characterizing motion of the person.

In implementations the method also involves obtaining a sequence of observations from the time series sensor data. In implementations the method further involves processing the sequence of observations to generate a representation of the sequence of observations as a two-dimensional image that represents relationships between the observations in the sequence. The two-dimensional image is processed using a (trained) artificial neural network comprising one or more convolutional neural network layers to generate classification data that predicts, for the sequence of observations, one of a plurality of categories.

In implementations the categories characterize different respective predicted motion states of the person. Examples of such motion states include a stationary motion state, a moving motion state, and a transitional motion state, e.g. static to moving (such as the person standing up or sitting or lying down), a falling motion state, a falling onset motion state (i.e. a state that indicates the onset of a fall), a fall risk motion state (i.e. a state that indicates that there is a risk of the person falling), and a neutral state (i.e. a baseline state). The predicted motion states may comprise one or more of these states.

The process may include, e.g., providing an alert in response to the generated classification data indicating that the person is in a particular motion state or has changed from one motion state to another.

In a further aspect there is described a computer-implemented method of detecting whether a person is exhibiting a heightened physiological response, more specifically detecting whether a person is exhibiting a biomarker of a heightened physiological response, in particular of sympathetic nervous system activation. The method involves generating, by measuring one or more physiological or motion parameters of the person using one or more sensors of a wearable device worn by the person, sensor data for the one or more physiological or motion parameters. Based upon the sensor data for each respective parameter the process determines one or more probability values indicative that a parameter within a first range corresponding to a first physiological or motion state will transition to a second range corresponding to a second physiological or motion state. The process can then generate input data based upon the one or more probability values. The process provides the input data as input to a machine-learning model to generate output data indicating whether the person is exhibiting a heightened physiological response.

In implementations the wearable device is in the form of a sock. The process can involve providing a recommended action or alert based upon the output data.

A wearable device for detecting whether a person is exhibiting a heightened physiological response can include the one or more sensors and a local or remote controller configured to perform a process as described herein.

There is also described a system for monitoring a person. The system comprises one or more physiological sensors to obtain physiological data characterizing a physiological state of the person, and/or motion sensors to obtain motion data characterizing motion of the person. The sensors may be provided on one or a pair of socks to be worn by the person.

The system also includes one or more computers storing processor control code that, when executed by one or more processors of the one or more computers, cause the one or more computers to implement a method as described above.

There is also described processor control code to implement the above-described systems and methods, for example on a general purpose computer system, or on a mobile device, or on a digital signal processor (DSP). The code can be provided on a non-transitory physical data carrier such as a disk, CD- or DVD-ROM, programmed memory such as non-volatile memory (e.g. Flash) or read-only memory (Firmware). Code and/or data to implement embodiments of the invention may comprise source, object or executable code in a conventional programming language (interpreted or compiled) such as C, or assembly code, or code for a hardware description language. As the skilled person will appreciate such code and/or data may be distributed between a plurality of coupled components in communication with one another. A machine learning classifier such as an artificial neural network as described herein can also be implemented in dedicated hardware, i.e. electronic circuitry, e.g. using an ASIC (Application Specific Integrated Circuit) or FPGA (Field Programmable Gate Array).

These and other aspects of the invention will now be further described by way of example only, with reference to the accompanying Figures.

### BRIEF DESCRIPTION OF THE DRAWINGS

FIG. 1 shows an example implementation of a system for monitoring a person.

FIG. 2 shows an example process for monitoring a person.

FIG. 3 shows use of a rolling window to obtain a sequence of observations.

FIG. 4 shows examples of two-dimensional Markov Transition Field images.

FIG. 5 shows an example Receiver Operating Characteristic and a confusion matrix.

FIG. 6 shows a wearable stress-detection device.

FIG. 7 shows a schematic representation of a stress type classifier.

FIG. 8 shows stress-related probability density functions.

FIG. 8 shows various stress and no-stress probability density functions.

FIG. 10 schematically illustrates a process for determining stress state and type.

FIG. 11 shows another example system to detect if a person is exhibiting stress.

FIG. 12 illustrates use of a stress detection system with adjustable sensitivity.

FIG. 13 schematically illustrates a sensitivity adjustment process.

FIG. 14 schematically illustrates a model training process.

FIG. 15 is a flow diagram of a method for detecting whether a person is showing stress.

### DETAILED DESCRIPTION

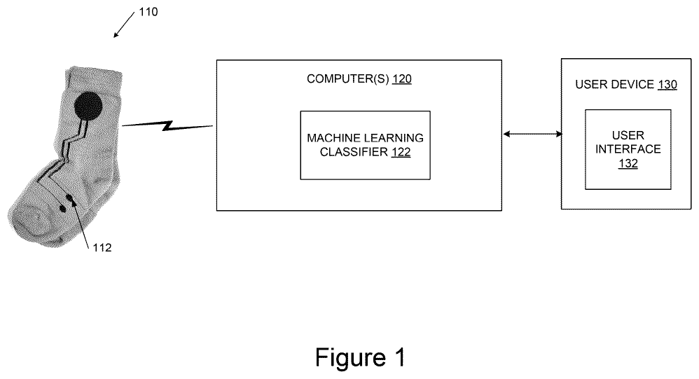

FIG. 1 shows an example implementation of a system 100 for monitoring a person, e.g. at their home, or in a residential care setting, or in hospital.

The system comprises a pair of socks 110 to be worn by the monitored person, at least one of which has one or more sensors 112. The socks can be washable; they can have a detachable part comprising, e.g., a power source and/or electronics for wireless connection to collect data from the sensors.

In implementations a sock is used to bring one or more of the physiological sensors into direct contact with the skin of the person to collect the physiological data from the foot or ankle. For example in implementations sock is used to hold the electrodes of an electrodermal activity sensor in contact with the skin, e.g. on the sole of the foot (which has a high density of sweat glands). It can also be useful to hold a motion sensor, such as an accelerometer or pressure sensor, in direct contact with the skin, to improve the accuracy of the collected motion data.

In implementations the one or more sensors comprise a sensor to sense electrodermal activity (EDA) of the skin of the person and a motion sensor such as an accelerometer, e.g. a 3-axis accelerometer or other IMU (Inertial Motion Unit). It has been found in practice that using a combination of an EDA sensor and a motion sensor can provide complementary information that can help stress detection.

The electrodermal activity sensor can sense an AC or DC skin response such as skin conductivity, skin impedance, or skin potential. For example the electrodermal activity sensor can sense one or both of slow varying tonic sympathetic activity (Skin Conductance Level, SCL) and fast varying phasic sympathetic activity (Skin Conductance Response, SCR).

In some implementations data from the three axes of a 3-axis accelerometer is combined to obtain a single acceleration value (ACT) by determining {square root over (Σᵢ₌₁³(a_{i,t}−a_{i,(t-1)}²)} where a_{i,t} is the acceleration data from axis i at time t.

Surprisingly it can be useful if the physiological data also includes data from a temperature sensor, such as an infrared thermopile, to sense the skin temperature (TEMP).

In some implementations heart beat data is also sensed, e.g. electrically or optically. This can be used to obtain a heart rate (HR) and/or an inter beat interval (IBI) or a version thereof e.g. a smoothed version of IBI, a linearly interpolated version of IBI (IBI_LERP), or an RMS (root mean square) version of IBI e.g. an RMS of successive differences (IBI_RMSSD). However in some implementations it can be advantageous to omit heart rate and related data as this can be affected by comorbidities such as heart arrhythmia, and treatments for these, or the presence of a pacemaker.

Some implementations of the system comprise one or more computers 120 that implement a machine learning classifier 122; in some implementations the machine learning classifier 122 can be partially or wholly implemented on dedicated hardware. In some configurations the machine learning classifier 122 is implemented on a remote server (in the cloud). In some configurations the machine learning classifier is implemented partly on wholly on a local computing device such as a mobile device, e.g. a mobile phone. This can facilitate privacy where, say, the machine learning classifier is fine-tuned using data specific to an individual. The socks 110 can have a wireless connection to the machine learning classifier 122, e.g. via an Internet router.

The system can also include a user device 130 such as a mobile device, e.g. a mobile phone, coupled to the one or more computers 120. This can implement a user interface 132, e.g. for collecting observed stress levels for fine tuning the machine learning classifier 122, or for providing alerts, or for setting a sensitivity of the system, or for interacting with the system generally. The system can also include other visual or audible alert devices (not shown in FIG. 1).

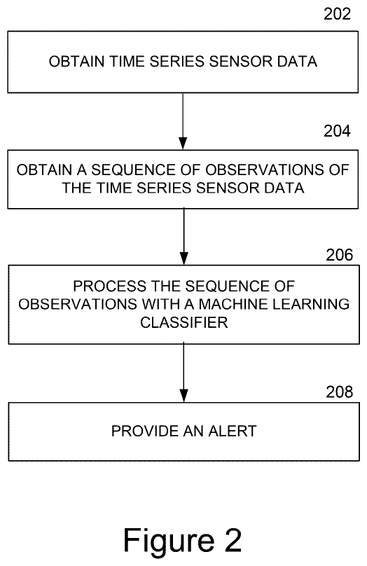

FIG. 2 shows an example process for monitoring a person, that can be implemented by a system comprising a wearable device and one or more computers in one or more locations, e.g. by the system of FIG. 1.

In an example implementation the process involves obtaining time series sensor data from one or more sensors on one or a pair of socks worn by the person (step 202). As an example the time series sensor data may comprise data sensed at intervals between 0.01 second and 10 seconds; e.g. a sampling frequency of order 50-100 Hz may be used.

The time series sensor data may be bundled into data packets that are buffered and transmitted at intervals, e.g. 1 minute interval, e.g. when a Wi-Fi connection is established. The sensor sample rates and transmission interval can be adapted to particular sensors and applications and may be configurable in software. In some implementations the raw sensor data is downsampled, e.g. to of order 1 Hz, in a pre-processing step prior to processing by the machine learning classifier.

The time series sensor data comprises physiological data from one or more physiological sensors characterizing a physiological state of the person, and/or motion data from one or more motion sensors characterizing motion of the person.

The system obtains a sequence or window of observations of the time series sensor data (step 204). An observation may comprise a (raw) sensor value at a particular time in the time series, or a processed version of one or more of the sensor values such as one or more values representing a normalized version of a sensor value, or one or more features representing the time series sensor data from a sensor. In general the sequence of observations may comprise any observations that characterize the time series sensor data. The sequence of observations can encode features of and/or relationships between multiple observations.

In some implementations the observations comprise normalized versions of the sensor data. For example, the data from one, more or all of the sensors can be normalized so that it has a mean of zero and/or a standard deviation of one. Normalization can be useful in putting the data from the different sensors into a shared range, which can facilitate achieving precision (so-called positive predictive value) in the classification data.

The sequence of observations is processed using the (trained) machine learning classifier to generate classification data (step 206). The classification data predicts, for the sequence of observations, one of a plurality of categories, e.g. that characterize different respective predicted states of stress of the person, or predicted motion states of the person as described further later.

Some implementations of the method include providing an alert in response to the generated classification data indicating a change in the predicted state of stress (step 208). The alert can be provided in any suitable manner, e.g. it may comprise an audible or visual alert and/or an alert sent to a mobile device of a carer or team.

The change in the predicted state of stress may indicate that the person has a high or heightened predicted state of stress (from amongst the categories of predicted stress states), and/or that the person has a low or lessened predicted state of stress, e.g. it may indicate a change to a higher or to a lower stress state. In some implementations, e.g. where an alert is generated on a particular detected level of stress, e.g. a high level of stress, an alert may only be generated after it has persisted for greater than a minimum duration, e.g. to selectively alert on more severe events.

A state of stress is not diagnostic of any particular disease. Nonetheless, if a heightened predicted state of stress is detected the person may be treated e.g. by talking to them, or asking them to perform certain actions, or by giving a drug to reduce stress.

In implementations obtaining the sequence of observations comprises applying a rolling window to the time series sensor data. This reduces the processing requirements by facilitating an incremental update of any features that are determined from multiple observations in the sequence.

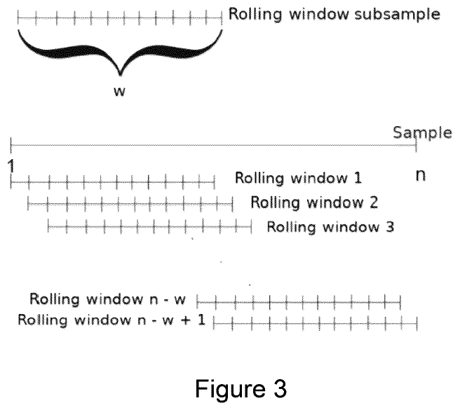

FIG. 3 shows, schematically, use of a rolling window to obtain a sequence of observations, w, of a succession of n samples of sensor data. In the illustration the sequence of observations, w, is obtained for successive time steps t=1,2,3.

Factors that influence the window length include the desirability of capturing local trends and anomalies, the timescales of changes in the sensor data, the desirability of timely responding to changes, and the desirability of reducing noise in the generated classification data. In implementations experiments suggest that the sequence of observations, e.g. the rolling window, can have a duration of at least 30 seconds or 60 seconds, e.g. of around 90 seconds.

In some implementations the sequence of observations is processed to generate a representation of the sequence of observations as a two-dimensional image that represents relationships between the observations in the sequence, e.g. a Markov Transition Field (MTF) or a Gramian Angular Field (GAF) representation of the sequence of observations.

The two-dimensional image is then processed using a (trained) artificial neural network, in particular (but not necessarily) one comprising one or more convolutional neural network layers, to generate the classification data. The neural network can in general include other layers, e.g. one or more batch norm or layer norm layers.

The classification data can comprise a score for each category of the plurality of categories, each score representing an estimated likelihood of a respective one of the states of stress. The category of the plurality of categories, here characterizing a predicted state of stress of the person, can be selected using the scores by, e.g., selecting the most likely category or by sampling from the distribution represented by the scores.

Techniques for determining a Markov Transition Field or a Gramian Angular Field representation of time series data are known and described, e.g. in Wang et al., arXiv:1509.07481, 2015.

In broad terms a Markov Transition Field can be obtained by determining Q quantiles of the values of the observations (i.e. the quantile of each value), and then determining a probability of a transition from quantile qᵢ to qⱼ (by counting the frequency of transitions from qᵢ to qⱼ), for each pair of quantiles, to populate a Q×Q matrix that is then treated as an image. The number of quantiles defines a granularity of the representation; merely as an example 10 quantiles can be used. The Markov Transition Field can be defined as:

$$M = [ \begin{matrix}{v}_{ij \vert {x}_{1} ∈ {q}_{i} , {x}_{1} ∈ {q}_{j}} & … & {v}_{ij \vert {x}_{1} ∈ {q}_{i} , {x}_{n} ∈ {q}_{j}} \\ {v}_{ij \vert {x}_{2} ∈ {q}_{i} , {x}_{1} ∈ {q}_{j}} & … & {v}_{ij \vert {x}_{2} ∈ {q}_{i} , {x}_{n} ∈ {q}_{j}} \\ ⋮ & ⋱ & ⋮ \\ {v}_{ij \vert {x}_{n} ∈ {q}_{i} , {x}_{1} ∈ {q}_{j}} & … & {v}_{ij \vert {x}_{n} ∈ {q}_{i} , {x}_{n}} ∈ {q}_{j}\end{matrix} ]$$

where xᵢ denotes an observation and vᵢⱼ denotes the normalized frequency with which a point in quantile qᵢ is followed by a point in quantile qⱼ.

In broad terms a Gramian Angular Field representation is determined by transforming a rescaled version of the time series (with values in the interval [−1,1] or [0,1]) into polar coordinates in which ϕᵢ=arccos \tilde{x}ᵢ and determining the Gramian Angular Field, G, as a matrix of with elements cos(ϕᵢ+ϕⱼ) that are each a form of inner product between values in the time series:

$$G = [ \begin{matrix}cos ( {ϕ}_{1} + {ϕ}_{1} ) & … & cos ( {ϕ}_{1} + {ϕ}_{n} ) \\ cos ( {ϕ}_{2} + {ϕ}_{1} ) & … & cos ( {ϕ}_{2} + {ϕ}_{n} ) \\ ⋮ & ⋱ & ⋮ \\ cos ( {ϕ}_{n} + {ϕ}_{1} ) & … & cos ( {ϕ}_{n} + {ϕ}_{n} )\end{matrix} ] .$$

MTF and GAF representations are examples of two ways in which the sequence of observations can be converted into a two-dimensional image that represents relationships between the observations in the sequence. Where there is time series data, and hence observations, from multiple sensors the time series data from each sensor can be represented in a respective channel of the image. An advantage to representing the relationships between the observations in the sequence is that in a rolling window based implementation the two-dimensional image can be incrementally updated as the window steps along the time series data. In some implementations updating the rolling window and updating the two-dimensional image can be performed in parallel, e.g. in a pipelined manner.

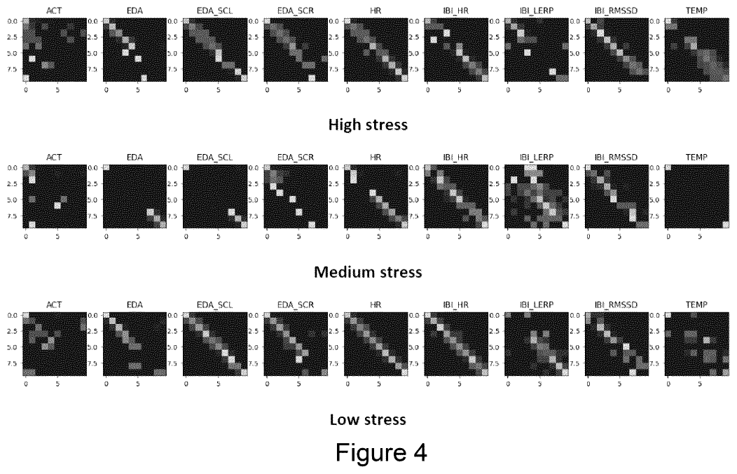

To illustrate this FIG. 4 shows examples of two-dimensional MTF images for low, medium and high stress. The example images have 9 channels with sensor data as described above. Generating a representation of the sequence of observations as a two-dimensional image that represents relationships between the observations in the sequence facilitates capturing local trends and anomalies as well as recognising dynamic transitions between states.

Implementations of the described techniques that use a neural network are not limited to any particular neural network architecture, or to any particular number of nodes and layers. For example it can include one or more feed forward, recurrent, convolutional, attention, or normalization layers. Where the sensor data is represented as a two-dimensional image the neural network should be configured to process an image that has a size and number of channels corresponding to the image. Where the neural network generates a score for each classification category it can have an output for each category. The number of layers can be chosen, e.g. as a trade-off between computational requirements and performance of the system.

Merely as an illustrative example, when the machine learning classifier comprises a convolutional neural network (CNN), the convolutional neural network may have the architecture below:

| Layer (type) | Output Shape | Param # |
|---|---|---|
| conv2d_16 (Conv2D) | (None, 10, 10, 32) | 2048 |
| batch_norm_1 (BatchNormalization) | (None, 10, 10, 32) | 128 |
| conv2d_17 (Conv2D) | (None, 10, 10, 32) | 9248 |
| batch_norm_2 (BatchNormalization) | (None, 10, 10, 32) | 128 |
| max_pooling2d_12 (MaxPooling2D) | (None, 5, 5, 32) | 0 |
| dropout_4 (Dropout) | (None, 5, 5, 32) | 0 |
| conv2d_18 (Conv2D) | (None, 5, 5, 64) | 18496 |
| batch_norm_3 (BatchNormalization) | (None, 5, 5, 64) | 256 |
| conv2d_19 (Conv2D) | (None, 5, 5, 64) | 36928 |
| batch_norm_4 (BatchNormalization) | (None, 5, 5, 64) | 256 |
| max_pooling2d_13 (MaxPooling2D) | (None, 2, 2, 64) | 0 |
| dropout_5 (Dropout) | (None, 2, 2, 64) | 0 |
| flatten_6 (Flatten) | (None, 256) | 0 |
| dense_12 (Dense) | (None, 64) | 16448 |
| batch_norm_5 (BatchNormalization) | (None, 64) | 256 |
| dense_13 (Dense) | (None, 2) | 130 |

In some implementations the sequence of observations is processed to generate a set of features or metrics representing the sequence of observations. In particular statistical metrics can be used such as one or more of a mean, median, standard deviation, and interquartile range of the sequence of observations, e.g. evaluated over a rolling window. Representing the sequence of observations in this way can have an advantage of sparsifying the data in the sequence.

In some implementations the set of features or metrics is processed using a decision tree model to generate the classification data. Such an approach can provide a reduced computational load compared to some other approaches.

Techniques for learning a decision tree are well known and there are several different approaches. In general a decision tree can be learned by working top down and choosing a best split for the data at each node based on some metric. There are many metrics that can be used. As one example the Gini index can be used (broadly, a measure of how often a randomly chosen element would be incorrectly labelled if it were labelled randomly and independently according to the distribution of labels), e.g. determined as G(t)=1−Σᵢ₌₁^{c}(pᵢ)² for C classes and a proportion pᵢ of instances belonging to a class i at a node t.

In some implementations the set of features or metrics is processed using a Naïve Bayes classifier to generate the classification data.

In general the predicted states of stress of the person can be categorised into two states, e.g. normal and elevated, or three or more states, e.g. low, medium, and high.

Some Implementations of the above described techniques the (trained) machine learning classifier comprises a (trained) artificial neural network having a plurality of neural layers and an output layer configured to apply a softmax function. Then the sequence of observations can be processed using the (trained) neural network to generate classification data comprising a score for each category of the plurality of categories, each score representing an estimated likelihood of a respective one of the states of stress.

The process can involve adjusting a temperature parameter of the softmax function to adjust the sensitivity of the monitoring, e.g. by obtaining a control input that specifies a sensitivity of the monitoring and using the control input to adjust the temperature parameter. For example the softmax function may have the form e^{-βz}^{ᵢ}/Z where β is the temperature parameter, zᵢ, is a score, and Z is a normalization factor. The control input may be obtained from a local or remote user interface. Here adjusting the “sensitivity” can refer to adjusting the shape of a distribution of the scores, and hence the shape of a distribution of the categories.

In some implementations the (pre-trained) machine learning classifier is adapted for use with a particular individual by further training or “fine-tuning” the machine learning classifier. That is the process may include obtaining (further) training data from a particular individual person and fine-tuning the trained machine learning classifier using the training data, before using the fine-tuned machine learning classifier to monitor the particular person. The fine tuning may involve supervised learning based on a labelled set of further training data. Sensor data for the (further) training data may be obtained from the sensors and labels for the (further) training data may be obtained based on self-reporting by the individual and/or based on reporting by a third party such as a carer.

The above described techniques can be used to process data from other types of sensor. For example in variants of the above described techniques the sensors are not part of a sock.

In some further variants, rather than using a (trained) machine learning classifier to generate classification data, a (trained) machine learning system, e.g. an artificial neural network, can generate a regression output that predicts a degree of stress of the person. For example in the case of a neural network this can be achieved by using a linear output layer (i.e. omitting the activation function) that generates a scalar output. The neural network can be trained by collecting training data generally as described herein but collecting the stress data as continuous rather than categorical data e.g. by rating the stress on a continuous scale rather as binned into discrete categories. That is, references to classifier may be substituted by references to a regression model.

In general a machine learning classifier, or another machine learning model or neural network as described herein, can be trained using data from a public database such as the open source Wearable Stress and Affect Detection (WESAD) dataset (Schmidt et al., “Introducing WESAD, a Multimodal Dataset for Wearable Stress and Affect Detection”, ICMI '18: Proceedings of the 20th ACM International Conference on Multimodal Interaction, October 2018 pages 400-408).

Also or instead physiological and/or motion data may be collected from a particular wearable device to be used in a system or process as described herein. Labels for the training data corresponding to the categories of classification may be obtained, e.g. from the wearer and/or a carer. The machine learning model may then be trained using a supervised training technique appropriate to the model.

As some further examples, data collected for training and/or fine tuning the system may also or instead comprise one or more of: an indication of whether the person is distressed; the severity of the distress; the kinds of behaviour exhibited; known triggers for the behaviour (if any); type of support provided to manage the behaviour; and whether the support was effective (obtained later). That is, this or other information reflecting the person's (wearer's) individual response or behaviour can be used to label the physiological and/or motion data. In implementations the volume of data available for training the system increases over time, and hence so can the classification accuracy.

In some implementations training data of this type can be obtained, e.g. in response to a request from the user device, e.g. in response to a prompt from an app running on a mobile phone, e.g. (but not necessarily) when the person is recognised as being stressed, agitated, or otherwise distressed.

As an example, for a system that includes a neural network to generate the classification data the model (neural network) may be trained using a cross-entropy objective (loss) or other classification objective (loss) function. In general training a neural network involves backpropagating gradients of the objective function to update learnable parameters, e.g. weights, of the neural network using a gradient descent optimization algorithm such as Adam. The training can continue until the loss plateaus; early stopping can be used. Training process can be evaluated based on held out examples from a training data set.

In more detail, when collecting training data from the sock(s) 110, data from the sensors may be time-aligned to a common sampling frequency, e.g. 1 Hz, and annotated with stress (or motion) labels corresponding to the categories, e.g. from the person and/or a carer. The training data may be but need not be collected from the same population as that with which the system will be used. With appropriate ethics stress can be induced, e.g. by asking a person to perform a mental arithmetic or memory recall task, optionally in the presence of a distractor such as a tone. As a particular example a Trier Social Stress Test (TSST) can be used. As another example to experimentally elicit stress a cold pressor test can be used (in a validated stress testing framework). Optionally outlying sensor data values can be discarded, e.g. by discarding sensor values (after normalization) that are beyond a threshold number of standard deviations (SDs) from the mean, e.g. 6 SDs.

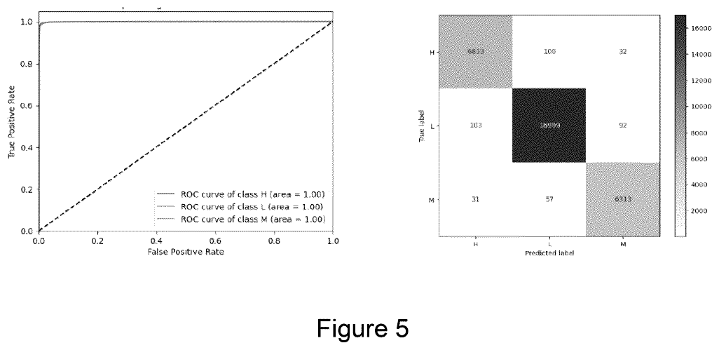

FIG. 5 illustrates a ROC (Receiver Operating Characteristic) curves and a confusion matrix for an implementation of the system using normalized sensor data with a 90 second rolling window to generate MTF images that are processed by a CNN including batch norm layers to generate a multiclass output indicating low, medium and high stress. These illustrate almost perfect performance on the held out training data.

The performance drops somewhat with new, unseen participants, i.e. with people that are not represented in the training data set. For this reason it can be useful to fine tune the system as previously described, e.g. for a period of a week or two, to adapt the system to a particular individual or individuals, based on self-reporting or carer-reporting. Optionally such fine tuning can be repeated at periodic intervals.

### Additional Examples and Variants

This specification also describes a wearable stress-detection device and a method for detecting the current stress state and stress type of the wearer. The results are outputted to a device, such as a smartphone or tablet, which provides one or more recommended actions to a carer to lower stress of the wearer of the device. The wearable device may, in other embodiments, provide recommended actions or coping strategies directly to the person wearing the device to support self-management and better regulation of distress and agitation (for people with autism and learning disabilities, for example). The device is intended to be used as a health care aid to assist carers of individuals with dementia or Alzheimer's disease. However, the device also has broader applications and could be used in the fitness industry to allow individuals to monitor their health.

A user can monitor the condition stress condition of a person, for example a carer can monitor the stress condition of a patient. A person may be both the wearer and user, i.e. monitoring their own stress condition using a phone configured to receive/determine the condition from sensors located on a wearer device.

Physiological parameters can include one or more of: heart-rate, heart rate variability, linear interpolation of heart rate, Electrodermal activity (EDA), EDA skin conductance response, EDA skin conductance level, skin temperature, and an activity index derived from accelerometer data, and in general any include parameter measurable by any sensor defined above.

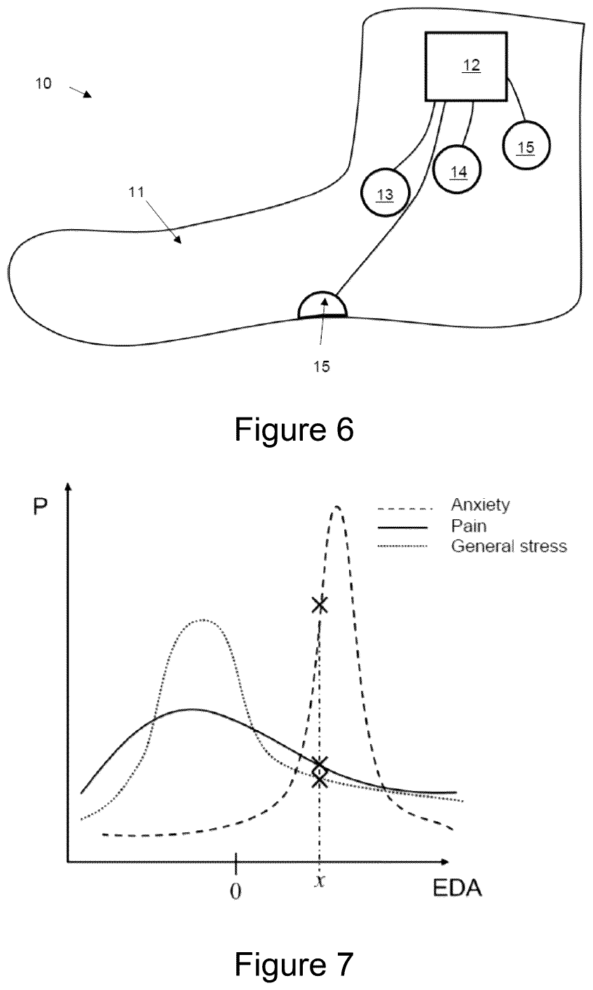

FIG. 6 shows a wearable stress-detection device 10 in the form of a sock 11. The sock 11 is made from an elasticated fabric material. The stress-detection device 10 comprises a plurality of sensors 13, 14, 15, 16 configured to obtain physiological data from the wearer. In the embodiment shown, the sock has a heart rate sensor 13, a skin temperature sensor 14, an electrodermal activity (EDA) sensor 15 (a galvanic skin response sensor) and an accelerometer 16. The heart rate sensors measure heart rate, which data can also be used to determine heart rate variability. The EDA sensor(s) measure EDA, which data can also be used to determine skin conductance levels and skin conductance response.

The sensors 13-16 are configured such that when the stress-detection device 10 is worn, the electrodes of the sensors are in close proximity to the wearer's skin, preferably in direct contact with the skin (i.e., the foot). The sensors 13-16 are connected to a controller 12. Each sensor is positioned in the most suitable locations for receiving signals. The EDA sensors are positioned at the arch of the foot, but could be positioned elsewhere on the sole of the foot, as the sole of the foot has a high density of sweat glands. The heart rate sensor is positioned at the ankle in this embodiment. The electrodes are made from a fabric material, for example a material comprising a conductive yarn.

The controller and a transmitter are located on a printed circuit board (PCB) which also accommodates a power source, e.g. a 3V lithium battery. An LED may be present on the PCB to indicate the device is operational. The PCB is attached to the inside of the sock using an attachment means, for example press fasteners. In some embodiments all the electronics may be integrally formed into the wearable device (e.g., moulded within). The sensors and controller can be at least partially hidden from view such as to be discrete. For example, the controller and sensors can be located on the inside of the foot such to be not visible.

The device comprises a transmitting means, which may be a wireless transmitter such as a WiFi (registered trademark) transmitter, or a Bluetooth (registered trademark) transmitter or other internet of things networking technologies. The transmitting means can be used for communicating the WiFi credentials from a smartphone or tablet. Data from the controller can be transmitted directly to a remote/cloud server over the internet, from which it can be transmitted to a device such as a smartphone, tablet or smartwatch. Alternatively the transmitting means may comprise a wired transmitter.

The circuitry on the PCB transmits the physiological parameter data collected from the sensors. In some implementations a remote/cloud server has memory storing the stress intensity classifier and stress type classifier for determining the stress state and type of the wearer.

The wearable stress-detection device facilitates continuous monitoring of physiological data of the wearer in a discrete manner. Integrating the controller and sensors into a wearable garment means the wearer is more likely to remember putting on the device. Socks are more familiar and less stigmatising than conventional wearables, such as watches, and can also be more difficult to remove. Significantly, the foot is a superior location for measuring EDA compared to the wrist due, e.g., to its relatively greater density of sweat glands. Nonetheless in some other implementations the wearable stress-detector may instead be a different garment such as a slipper, trousers, t-shirt, gloves; or a smart-watch.

In implementations the wearable stress-detection device determines the current stress state and stress type of the wearer using stress intensity and stress type classifiers respectively. In implementations the stress-detection device comprises a stress type classifier for determining the type of stress experienced by the wearer. In implementations the stress type classifier can differentiate between three different stress types: specifically pain, anxiety or other generalised non-specific stress (i.e. stress that is not caused by pain or anxiety).

In implementations the sensors on the wearable stress-detection device measure physiological parameters of the user, e.g. heart rate (HR), EDA, and skin temperature, and generates an input variable for each of these parameters. Heart rate variability, and in particular the root mean square successive difference (RMSSD), is calculated using the inter-beat interval (IBI). For example,

$$heart rate = Input\_HR \\ heart rate variability = Input\_HR \_RMSSD \\ electrodermal activity = Input\_EDA \\ skin temperature = Input\_TEMP$$

The stress type classifier can use a machine learning approach for determining the stress type. For example Naïve Bayes can be used to predict the correct stress type being experienced by a person. As an illustration, for each stress type, e.g. pain and anxiety, there is a probability density function per each physiological parameter. The probability density function represents the probability of the person experiencing a stress type for a given input variable, e.g. physiological parameter. For four physiological parameters being monitored there can be twelve probability density functions.
Other_EDA(x), Pain_EDA(x), Anxiety_EDA(x),
Other_HR(x), Pain_HR(x), Anxiety_HR(x),
Other_HR_RMSSD(x), Pain_HR_RMSSD(x), Anxiety_HR_RMSSD(x),
Other_Temp(x), Pain_Temp(x), Anxiety_Temp(x),

The stress type classifier evaluates the input variable generated for each physiological parameter against the three stress types for which its associated. For example, the Input_EDA input variable is evaluated with reference to the Other_EDA, Pain_EDA and Anxiety_EDA probability density functions. The Input_HR input variable is evaluated by the Other_HR, Pain_HR, Anxiety_HR probability density functions and so forth. An intermediate variable is generated by each probability density function. For example,

$$Other\_EDA ( Input\_EDA ) = Intermediate\_EDA \_Other \\ Anxiety\_EDA ( Input\_EDA ) = Intermediate\_EDA \_Anxiety \\ Pain\_EDA ( Input\_EDA ) = Intermediate\_EDA \_Pain$$

A pain probability score can be calculated by multiplying all the intermediate variables generated by all the pain probability density functions together (e.g., Intermediate_EDA_Pain×Intermediate_HR_Pain× . . . ). Similarly, anxiety and generalised non-specific stress probability scores can be calculated by multiplying together the respective intermediate variables generated by the anxiety and generalised non-specific stress probability density functions.

In implementations the stress type classifier compares the pain, anxiety and general stress probability scores to determine which is the most likely stress type of the wearer. A higher/highest probability score corresponds to a more/most likely stress type.

A stress type output signal is generated and can be transmitted to a device indicating the stress type.

In implementations the stress type classifier assumes a positive stress state of the person, which can be determined using a stress intensity classifier. If the person is not determined to be stressed, the stress type classifier need not be activated or the stress type output signal can be ignored.

In some implementations the stress type classifier also determines the stress state of the person independently, i.e. without the stress intensity classifier. For example, where the probabilistic score of one of the stress type is above a pre-selected value, the stress type classifier may assume both the stress state and stress type of the person and generate a stress type output signal accordingly.

In some implementations, the stress type classifier learns from, and may be continually retrained on, observations of stress recorded by a carer, e.g. recorded using a smartphone, tablet or other device.

FIG. 7 shows a schematic representation of a stress type classifier that returns a most probable stress type based on three known probability densities, one for each stress type, represented as curves. The x-axis is EDA measured in standard deviations (from a mean); the y-axis corresponds to the probability of the stress type for a given input variable value (x). The EDA data is standardised (normalized) prior to determining the probability density functions such that the mean value is at 0 and the standard deviation is at 1, facilitating comparisons between different individuals.

As shown in FIG. 7, all three probability density functions are represented as bell curve/Gaussian distributions. The anxiety probability density function has positive mean, modes and median values. Both the pain and generalised non-specific stress probability density functions have slightly negative mode, mean and median values. A process for generating the probability density functions is described below.

For a standardised input variable ‘x’ representing an EDA measurement taken from the wearer, the probability of a stress type can be determined by reading the values from the y-axis where the input variable intersects with each of the three probability density functions. In the specific example shown in FIG. 7, the probable stress type for the given EDA input variable ‘x’ is anxiety as this has the largest probability value for this measurement. This probability score for social anxiety can be generated as a stress type output signal and can be transmitted to a device indicating that an anxiety stress has been determined.

FIG. 7 only shows the probability based on a single physiological parameter, i.e., EDA. Other physiological parameters may indicate another stress type is more probable. To improve the accuracy of the classifier, a plurality of physiological parameters can be taken into consideration.

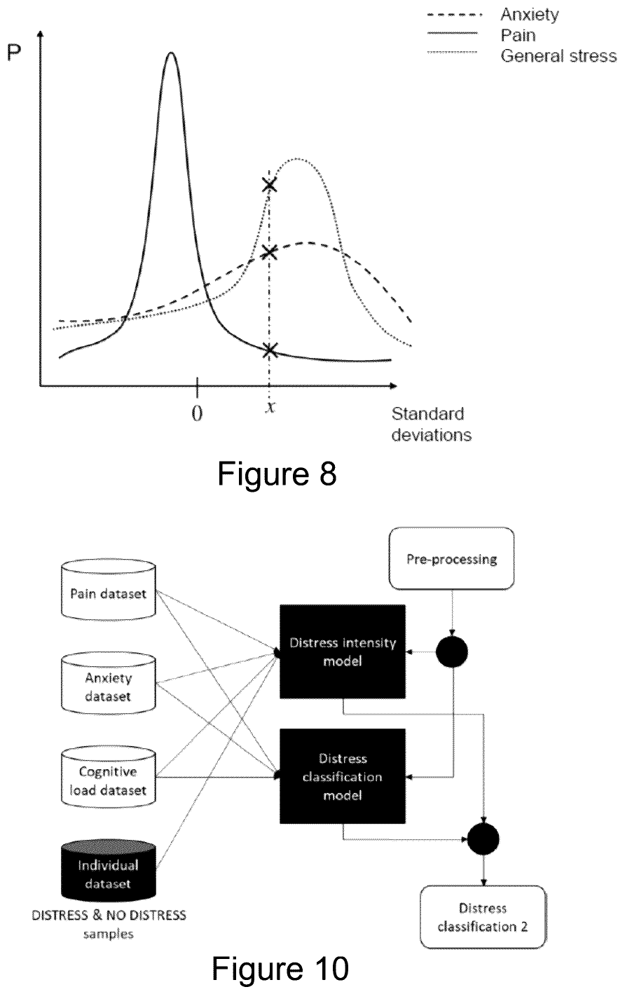

FIG. 8 shows pain, anxiety and generalised non-specific stress probability density functions plotted for skin temperature measured in ° C. Comparing FIGS. 7 and 8 shows that the stress types have different probability density functions across different parameters. The pain probability density function has lower mode, mean and median values than the corresponding values for the probability density functions of the anxiety and generalised non-specific stress types. The data used to compile the three probability density functions has been standardised. In the specific example shown in FIG. 8, for the measured skin temperature input variable ‘x’, the most probable stress type is generalised non-specific stress.

The stress type classifier makes a similar determination across all the measured physiological parameters. To weight the probability values generated by each of the stress type probability density functions together, the stress type classifier assigns the probability values generated by the probability density functions to intermediate variables. The likelihood of pain is calculated as the product of the conditional pain probabilities across all the physiological parameters. e.g., the probability value generated by the EDA pain probability density function is multiplied by the probability value generated by the skin temp pain probability density functions and so forth. Similar calculations are made for the anxiety and generalised non-specific stress probability values. The stress type classifier determines which stress type has the highest probability across all the measured parameters and generates a stress type output signal to the person or carer. The stress type output signal communicates the most probable stress type with a confidence estimate.

The probability density functions used by the stress type classifier can be derived through experimentation to find specific relationships between specific stress type and physiological parameters, such as to find relative differences which can be exploited. To achieve this, the relationship between a measured physiological parameter and a probability of each stress type can be determined.

Physiological measures exhibit high inter-subject variability due to individual differences in physiology and psychology. To make useful comparisons, raw measurement data can first undergo a pre-processing stage to standardise (e.g. normalize to zero mean unit variance) data and remove potential outlier datapoints. Any datapoints which are a located beyond predetermined values can be deemed to be outliers and rejected from the sample. The standardisation process can occur prior to any training and prediction processes.

Table A shows characteristic data of the probability density functions. The data is compiled from four experiments, two testing cognitive load (i.e., non-specific stress) (conditions 1-3 & 4-6), one testing pain (condition 7, e.g. using ice cold water) and one testing anxiety (condition 9) forming four datasets, conditions 1-3, 4-6, 7 and 9. The participants of each condition were asked to report ‘change in state’, which could be binary (e.g., stressed or not stressed; pain or no pain; relaxed or not relaxed), a linear scale (e.g., 1-10) or any other appropriate self-reporting methods for the condition tested. In the example the participant's self-reported responses were assigned as either ‘stressed’ or ‘not stressed’.

The values in Table A show the deviation from the standardised mean for each physiological parameter (and each condition).

TABLE A

| Conditions | | EDA | EDA_SCL | EDA_SCR | HR | RMSSD_HR | IBI_LERP | TEMP |
|---|---|---|---|---|---|---|---|---|
| Cognitive | 1 | 0.097964 | 0.093688 | 0.093937 | 0.317193 | 0.325583 | −0.291422 | 0.106474 |
| load | 2 | 0.093897 | 0.112567 | −0.128161 | 0.043041 | −0.140662 | 0.118125 | 0.259477 |
| (generalised | 3 | 0.131889 | 0.140404 | −0.010308 | 0.263441 | 0.157066 | −0.145451 | 0.242024 |
| non- | 4 | 0.108863 | 0.106904 | 0.028392 | 0.233438 | 0.043237 | −0.038167 | −0.066167 |
| specific | 5 | −0.054407 | −0.063797 | 0.032573 | 0.135916 | −0.061993 | 0.060372 | −0.001072 |
| stress) | 6 | 0.036304 | 0.028738 | 0.040644 | 0.246236 | −0.012988 | 0.015243 | −0.044826 |
| Pain | 7 | −0.143455 | −0.180096 | 0.094694 | −0.131567 | −0.05437 | 0.080452 | −0.514071 |
| (Social) | 8 | 1.024749 | 0.815155 | 0.661255 | 0.872854 | 0.788485 | −0.712592 | −0.021731 |
| Anxiety | 9 | 1.427375 | 1.19832 | 0.814252 | 1.215021 | 1.204544 | −1.061057 | 0.071021 |

Notable distinctions for the stress types can be seen for the physiological parameters in each of the columns. Specifically:
Individuals experiencing social anxiety exhibit higher EDA, EDA SCL, EDA SCR, HR and IBI HR readings than those experiencing either pain or cognitive load stresses, typically between 0.8-1.2 standard deviations.
Individuals experiencing social anxiety exhibit lower IBI LERP readings than those experiencing either pain or cognitive load stresses, typically between 0.7-1.1 standard deviations.
Individuals experiencing pain exhibit more negative skin temperatures than those experiencing either cognitive load or social anxiety stresses, typically around 0.5 standard deviations.

The values in the table are used to form Gaussian distributions which form the probability density functions used in the stress type classifier.

The values in the table are indicative of the relative differences between the probability density functions (i.e. not absolute values). For example, conditions 1-9 show the mean temperature difference of a person experiencing pain is likely to be around 0.5 standard deviations lower than the same person experiencing either cognitive load or social anxiety stresses.

While the values in the table show the deviation from the mean standardised values, the relative differences also translate to similar differences to the mode and median of the probability density functions.

The experiments were conducted under laboratory conditions on 71 participants, who were mostly older people. Each condition 1-9 was purposely designed to elicit a specific stress type (e.g., pain, anxiety, and generalised non-specific stress). Participants were fitted with EDA, heart rate and temperatures sensors, amongst others, before the experiment started.

They then were told to perform a task specific to the condition tested and indicate when they felt changes in state. For each condition, the self-reported responses were labelled being either ‘stress’ or ‘not stressed’.

The data is represented in ‘Graphs A, B and C’ by way of example. The left of graph of ‘Graph A’ shows median distribution of participants for condition 1 when self-reporting either ‘stress’ or ‘not stressed’.

Table B shows the difference between the mean of the interquartile range of the stress and not/not stress self-reported data. The same method of finding relative differences between the physiological parameters for the stress types applied above can be used such that the stress type classifier can identify the particular stress type of a person.

TABLE B

| Conditions | | ACT | EDA | EDA_SCL | EDA_SCR | HR | RMSSD_HR | IBI_LERP | IBI_RMSSD | TEMP |
|---|---|---|---|---|---|---|---|---|---|---|
| Cognitive | 1 | 0.037521 | −0.0095 | −0.01434 | 0.068714 | −0.10931 | 0.052557 | 0.025849 | 0.051053 | 0.010862 |
| load | 2 | 0.052364 | 0.010988 | 0.001753 | −0.05067 | 0.024109 | 0.024137 | 0.038663 | 0.172972 | 0.020468 |
| (generalised | 3 | 0.060795 | −0.00019 | −0.00959 | 0.019463 | −0.06638 | 0.054648 | 0.043632 | 0.146555 | 0.020928 |
| non- | 4 | 0.030957 | 0.022495 | 0.022792 | 0.004159 | 0.057601 | 0.064159 | 0.052266 | 0.062048 | 0.00607 |
| specific | 5 | 0.035607 | 0.017538 | 0.018921 | 0.032852 | 0.057544 | 0.06176 | 0.042613 | 0.086843 | 0.018848 |
| stress) | 6 | 0.044376 | 0.026689 | 0.027809 | 0.024674 | 0.076763 | 0.083565 | 0.062925 | 0.098989 | 0.016612 |
| Pain | 7 | −0.1066 | −0.0482 | −0.08695 | −0.04521 | −0.05958 | 0.082785 | 0.047568 | 0.078759 | 0.027428 |
| (Social) | 8 | 0.396179 | 0.060352 | 0.18198 | 0.598753 | 0.228578 | 0.079449 | −0.28591 | −0.055041 | −0.00148 |
| Anxiety | 9 | 0.516845 | 0.097199 | 0.232082 | 0.722838 | 0.30128 | 0.130746 | −0.41684 | −0.058617 | −0.00229 |

As with Table A, notable distinctions for the stress types can be seen for the physiological parameters in each of the columns. Specifically:
Individuals experiencing anxiety stress have accelerator (ACT) readings than those experiencing a generalised non-specific stress or pain, typically between 0.4-0.5 standard deviations.
Individuals experiencing anxiety stress have higher EDA skin conductive response levels than those experiencing a generalised non-specific stress or pain, typically between 0.6-0.7 standard deviations.
   Individuals experiencing anxiety stress have lower IBI LERP levels than those standard deviations.

In relation to the above, in one approach four experiments divided into various conditions were conducted. The participants of each condition used a self-reporting method appropriate for that the condition to denote a change in their current state. The state could be mood, relaxation level, pain level etc., but here the denoted change state was a binary change between stress and not/no stress. Values from sensors measuring physiological parameters of the participants were then mapped to the self-reported data, enabling analysis of how the physiological parameters are affected by the participants stress state.

For conditions 1-3, the participants were asked to perform mental tasks of increasing difficulty specifically designed to challenge their cognitive abilities. The first condition was to repeatedly select the highest values in a 4×4 number grid over a three-minute duration. The condition task had two actions to complete; a Stroop Colour Word (SCW) test that required matching colours to their word counterparts, and an attention task which involved preventing a dot from leaving a target onscreen. The time allocated to the second condition was five minutes and was designed to provide moderate cognitive stress. The third condition was designed to induce a high level of stress. It involved two tasks: a mental arithmetic task and an attention task which required reporting all instances of a high-pitched audio tone that was interspersed with a low-pitched tone.

For conditions 4-6 a different set of participants were asked to perform mental tasks of increasing difficulty to challenge cognitive ability. Condition 1 was designed to induce a low level of stress. It involved a single task: copying a telephone number onscreen using either the keyboard or mouse. Condition 2 was designed to induce a moderate level of stress. It involved multiple tasks which had to be completed simultaneously: clicking all instances of the highest number in a 4×4 number grid, reporting all instances of a high-pitched audio tone which was interspersed with a low-pitched tone, and an attention task which involved preventing a dot from leaving a target onscreen. Condition 3 was designed to induce a high level of stress. It involved multiple tasks which had to be completed simultaneously: recalling a list of letters from memory, undertaking mental arithmetic, and an attention task which required periodically resetting a set of bars onscreen. Condition 7 was designed to test the participants pain response under ten different conditions. The first eight situations were designed to simulate activities of daily living:
situation B involved rising from bed
situation C involved performing gentle exercises, guided by an instructional video
situation D involved reading a magazine whilst sitting down on the sofa
situation E involved watching television whilst sitting down on the sofa
situation F involved preparing a cup of tea
situation G involved buttering a slice of bread
situation H involved putting on a jacket
situation I involved removing the jacket

The remaining two situations were purposely designed to be more stimulating:
situation J was designed to induce positive stimulation or excitement. It involved interacting with a robotic pet called Miro (Consequential Robotics).
situation K was designed to induce negative stimulation or stress. It involved performing a cold pressor test, a widely used method of experimental pain induction (Lovallo, 1975). The participant was asked to submerge their non-dominant hand in a bucket of ice-cold water for a period of 3 minutes.

Conditions 4-6 were set up to elicit a cognitive load stress type, which can be considered as a type of a generalised non-specific stress. The measured physiological parameter readings of cognitive load and a generalised non-specific stress are consistent, particularly in respect to the other measured stress types (i.e., the characteristics differences between cognitive load and generalised non-specific stress are the same or broadly the same when compared to the pain and anxiety stress types).

Conditions 8-9 were designed to measure the social anxiety of the participants during four situations:
The Baseline situation had a 20-minute duration and was designed to induce a neutral affective state. Subjects were seated at a table with access to neutral reading material (magazines).
The Amusement situation had a seven-minute duration and was designed to be enjoyable. Subjects watched a set of eleven funny video clips, each followed by a short neutral sequence of five-second duration.
The Stress situation had a ten-minute duration and was designed to induce high levels of stress. Subjects were exposed to the Trier Social Stress Test (TSST) (Kirschbaum et al., 1993), which consists of a public speaking task followed by a mental arithmetic task. The socially-evaluative and cognitively challenging elements of the TSST have been shown to elicit a reliable stress response (Plarre et al., 2011). Participants were allowed three minutes to prepare a five-minute speech on their personal traits, strengths and weaknesses to a three-personal panel of human resources specialists, however their notes were removed before they gave the speech. Afterwards, subjects were asked to count down from 2023 to zero in steps of 17, in a five-minute mental arithmetic activity, starting over when they made a mistake. Upon completing the TSST, participants rested for ten minutes.
The Meditation situation had a seven-minute duration and was intended to return subjects to a neutral affective state. It involved a controlled breathing exercise administered through an audio track, which participants performed with eyes closed whilst comfortably seated.

Conditions 8-9 were set up to be elicit a social anxiety stress type which can be considered as a specific type of anxiety stress. The measured physiological parameter readings of social anxiety and anxiety are consistent, particularly in respect to the other measured stress types (i.e., the characteristics differences between social anxiety and general anxiety are the same or broadly the same when compared to the pain and non-generalised stress types i.e., cognitive load).

The standardised results for the participants were collated together for each experiment with ‘stress’ and ‘not stressed classifiers’ tags once the experiments were completed.

Referring to FIG. 9, Graph A shows the stress and no stress features for EDA of condition 1. The left graph shows the standardised median data for stressed and no-stressed self-reported data (i.e., when the participants indicated a change in stress state during the experiment). The right graph represents changes in the inter-quartile ranges between from the same condition (i.e., condition 1).

The stress and no stress plots/features for both the median and inter-quartile ranges show no discernible differences. The value in condition 1—EDA cell in table A shows the mean difference between median stress and non-stress features to be 0.097964 (i.e., relatively close to zero).

The values in Tables A and B are determined by finding the mean of each of the stress and no stress curves (i.e., datasets) and subtracting them.

The difference between the median stress and non-stress features for the other conditions measuring generalised non-specific stress (i.e., conditions 1-6) shown in Table A but the graphs are not shown for conciseness. However, it can be seen the difference between the mean values is close to zero in each case.

Graph B shows the experimental data collected from condition 7 corresponding to observed changes in the skin temperature of the participants. The ‘non-stress’ feature for the median plots was-0.514071 standard deviations less than mean of the ‘stress’ feature. There were no notable differences observed between the mean differences of the median ‘stress’ and ‘non-stressed’ features for condition 7 for EDA, EDA SCL, EDA, HR, IBI HR and IBI LERP.

Graph C shows the median and inter-quartile plots for EDA from condition 9. The mean of the ‘stress’ feature was found to be 1.427375 standard deviations greater than the ‘non-stress’ feature. This graph is representative across conditions 8 and 9 for EDA, EDA SCL, EDA SCR, HR and IBI HR. However, the stress feature for IBI LERP showed a lower value for both experiments 8 and 9 being −0.712592 and −1.061057 respectively. There was no noticeable difference observed between the ‘stress’ and a ‘non-stress’ features for the skin temperature for experiments 8 and 9.

The relative differences between the values for a physical parameter against each condition in Table A provides a means for distinguishing particular stress types from one another (which can be used by the stress type classifier). The values can be used as the standardised deviation for Gaussian distribution curves used by the stress type classifier (i.e., probability density functions). For example, the EDA generalised non-specific stress probability density function is a Gaussian distribution with a standard deviation in the range of −0.05-0.04; the EDA pain probability density function is a Gaussian distribution with a standard deviation around of −0.14; the EDA anxiety probability density function is a Gaussian distribution with a standard deviations in the range of 1.02-1.42.

The standard deviation determines other characteristics of the probability distribution functions which can be illustrative of the relative differences between the curves. The probability density functions need not be (or be modelled as) Gaussian distributions. However, the relative difference characteristics of the curves (such as mean, mode, median) are still useful metrics.

Some implementations of the stress-detection device uses a stress intensity classifier to determine the stress state of the wearer. The stress state can be represented as a numerical value indicating a ‘confidence’ or probability score of the stress state. Alternatively, it can be represented as ‘LOW, MEDIUM or HIGH’ ordinal values, or even in binary form (e.g., ‘stressed’ or ‘not stressed’). This could involve a combination of a methods, for example, several numerical intervals could be used to represent LOW, MEDUIM and HIGH scores.

The stress intensity classifier can determine a person's stress state using a binary decision tree which uses the physiological parameters as conditional statements which determine if the wearer's measured data is within a preselected range for the heart rate, heart rate variability, EDA, and skin temperature etc. The ranges may be any of mean, mode, median and/or inter-quartile ranges. For example, a conditional statement may state a heart rate within the range of 60-70 beats per mins equates to the person not being stressed. Deviation beyond the range boundaries indicates the person is possibly stressed and the decision trees moves onto the next conditional parameter. Instead, or in addition, the stress intensity model can use other methods for determining the stress state, such as Naïve Bayes.

Some implementations of the stress intensity classifier use an individual dataset which contains baseline data readings of the wearer taken during a training phase. This can involve a period of self-reporting or reporting by a third party such as a carer. Physiological parameter readings are taken from the wearer over a period of time and are tagged (labelled) as either ‘stressed’ or ‘not stressed’.

The reporting can be done through a smart device application where the wearer or a carer indicates whether the care recipient (i.e. wearer of the device) is stressed or not stressed for a specific period of time. The application presents e.g. two options mapping onto stressed and non-stressed states. The application may assume the care recipient is in a particular stress state until otherwise indicated by them or the carer. For example, the device may assume the person e.g. care recipient to be unstressed unless otherwise indicated. The application may estimate the stress state when a change has occurred in one or more of the physiological parameters; for example, when a parameter transitions beyond a preselected value.

Furthermore, the individual dataset is used to update the conditional operators of the decision tree such that the conditions for determining the stress state of the care recipient are more accurately reflected. For example, two individuals may have significantly differing levels of fitness. An overweight person may have both a higher mean heart rate and heart rate variability, meaning their heart rate may be subject to a sudden drastic change even while not feeling stressed. This may be represented on the condition tree as a non-stressed conditional operator having a wider range. A younger fitter person may have a lower heart rate inter variability and therefore a narrower range.

Once the stress state for the wearer is determined, a second output signal is generated and transmitted to a smart device.

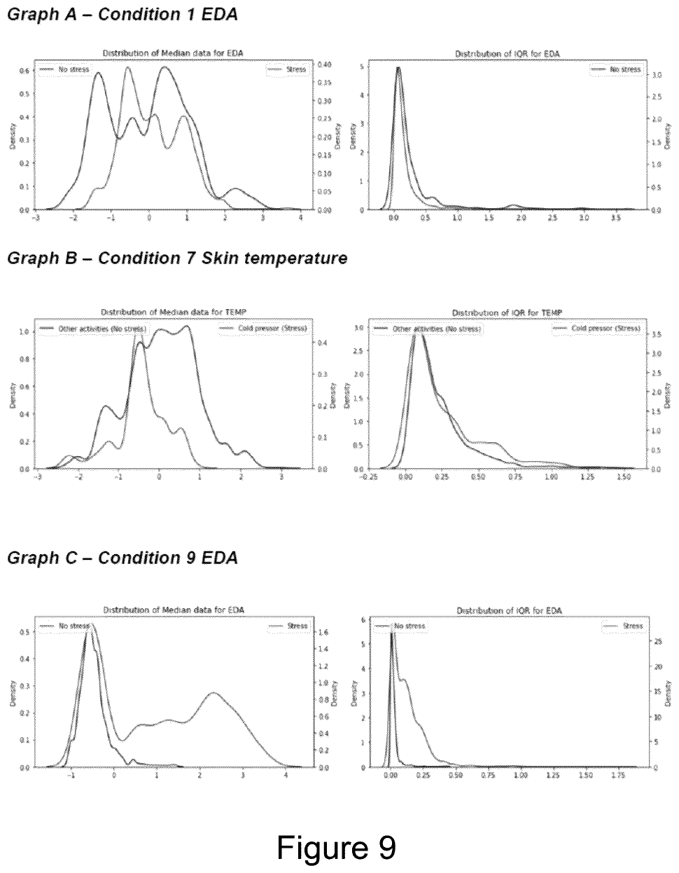

FIG. 10 illustrates, schematically, an overall process for determining the stress state and stress type. Physiological data measured from the wearer undergoes a pre-processing stage in which the data is standardised, and any outliers removed. The data is assigned to input variables and transmitted to both the stress intensity and stress type classifiers. The pain, social anxiety and cognitive load datasets contain the experimental data explained above and are used to compile the decision tree values and/or ranges used in the stress intensity model, as well as the probability density functions used by the stress type classifiers. An individual dataset contains wearer specific information which is gained during the initial training phase and is used by the stress intensity classifier. Optionally the individual dataset can be continuously updated throughout operation of the system. The individual dataset can be used to adjust/refine the conditional ranges of the decision tree of the stress intensity classifier such to give more accurate results. This changing of the conditional ranges can be used as a sensitivity adjustment.

The stress intensity model can generate a stress intensity output signal indicating the determined stress intensity or stress state. The stress type classifier can generate a stress type output signal indicating which stress type has been determined.

One or both output signals are transmitted to a processor, which collates the signals to present to a readable form to the wearer and/or carer. This processing can occur locally on the controller of the wearable stress-detection device, in the cloud or a on the smart device on which it is displayed. If the model determines the wearer not to be stressed, the second output signal can be ignored.

In some implementations the processor contains a list of pre-selected actions which a carer can perform to lessen the stress the wearer. For example, if the wearer is determined to be stressed, the list of recommended actions may include one or more of: tell the wearer to take a break from their current activity; take the wearer for a short walk; distract the wearer with personal item; verbally engage with the wearer.

To summarise, described above is a method of detecting a stress condition of a person, comprising measuring one or more physiological parameters of the person to generate an input variable for each of the one or more physiological parameters, using a stress type classifier to evaluate the one or more input variables to determine a type of stress being experienced by the user, selected from a plurality of pre-determined types of stress, and generating a stress type output signal corresponding to the determined stress type.

Also described is a device for detecting a stress condition of a person, comprising one or more sensors for measuring one or more physiological parameters, and a controller configured to perform the method for detecting a stress condition of a person. The stress condition can be exhibited as either an indication that a person is stressed or not stressed, and/or experiencing a specific stress type.

Here ‘stress intensity’ can refer to a determination of a stress state of a person. As an example it can be represented as an intensity level, e.g. on a numerical scale, where a higher score corresponds to a higher stress. Also or instead the stress intensity level may represent a confidence score that the person is stressed, e.g., a higher score being indicative of an increased likelihood of the person being stressed.

The stress intensity may be represented as two or more states. In the simplest form, the stress intensity may be represented as a binary ‘stressed’ or ‘not stressed’ state; or as ‘LOW’, MEDIUM or HIGH states. It may also be a combination of the above representations. For example, ranged intervals within a stress intensity level range may corresponds to a specific state. For example, LOW stress state being at or below a first value, MEDIUM stress state between the first value and a second value, and HIGH stress state being at or above a third value.

References to stress type can mean a particular type of stress, e.g. that has a defined cause or causes. For example, stress types may include a pain stress type, i.e. stress caused by pain, or an anxiety stress type, i.e. stress caused by anxiety, which are both specific types of stress with specific causes. The stress types may also include an unspecified or general stress, which may be defined as stress that has not been classified as the one or more specific stress types, e.g. not classified as a pain stress type or an anxiety stress type.

The stress condition of a person may be found using one or more physiological parameters using sensors. The sensors may be, e.g., any one or more of the following: heart rate sensors, Electrodermal activity (EDA) sensors, temperature sensors, magnetic field sensors, optoelectronic/optical sensors, pressure sensors, global Navigation Satellite System (GNSS) sensors, or motion sensors. The heart rate sensor may measure one or more physiological parameters related to heart rate, e.g., heart rate (e.g. bpm), heart rate variability and/or linear interpolation of heart rate. The EDA sensor may measure one or more physiological parameters related to EDA, e.g., EDA, EDA skin conductance response and/or EDA skin conductance level. The temperature sensor may measure the skin temperature of the person. The magnetic field sensor may be a magnetometer which may measure the person's gait. The optoelectronic or optical sensor may be a camera or pulse oximeter which may measure one or more physiological parameters related to the person's bloodwork e.g., (non-invasive) blood oxygen measurements. The pressure sensor may be a blood pressure monitor (e.g., used on an ankle cuff) or a piezoelectric or other pressure sensor (e.g., which may be located on the soles for estimating the wearer's centre of mass for fall detection) for measuring one or more physiological parameters related to pressure. The GNSS may be a global positioning system (GPS) sensor for determining the position of the person. The motion senor may be an accelerometer, e.g. that derives an activity index derived from accelerometer data; or gyroscope which may measure the person's gait. The physiological parameters may be a combination of the groups, e.g., a wearable device may comprise a heart rate sensor for measuring two physiological parameters, e.g., heart rate and heart rate variability, an EDA sensor for measuring one physiological parameter such as EDA and a temperature sensor for measuring one physiological parameter such as skin temperature.

Some implementations of the method use a stress type classifier for determining the type of stress being experienced by the person, e.g. by determining which of the pre-determined types of stress is most likely using appropriate statistical analysis. The stress type classifier may can take one or more measured physiological parameters and provide a stress type output signal, which corresponds to a type of stress being experienced by the person.

The stress type classifier may be a machine learning classifier, e.g. a Naive Bayes classifier. The stress type classifier may be pre-trained with labelled data, which may enable the stress type classifier to exploit statistical differences in the physiological parameters with respect to different stress types (e.g. pain and anxiety) to output the most probable stress type (e.g. the maximum likelihood estimate) as one of the pre-determined types of stress. Alternatively, the stress type classifier may, e.g., use a decision tree to determine the stress type.

Experiments may be conducted to determine how the physiological parameters of participants in the experiments change when incited to experience different stress types, and the resultant data may be used to pre-train the stress type classifier. Experiments may be specifically configured to incite a particular type of stress in the participants. For example, to induce a pain stress type, the participant may induce pain, e.g. by placing their hand in a cold water bucket, and then self-report their current stress state. The self-reported data may be mapped to the physiological parameter data for the participant that was measured during the experiment. Each physiological parameter measured may therefore be labelled with the self-reported stress state, e.g. stressed or not stressed.

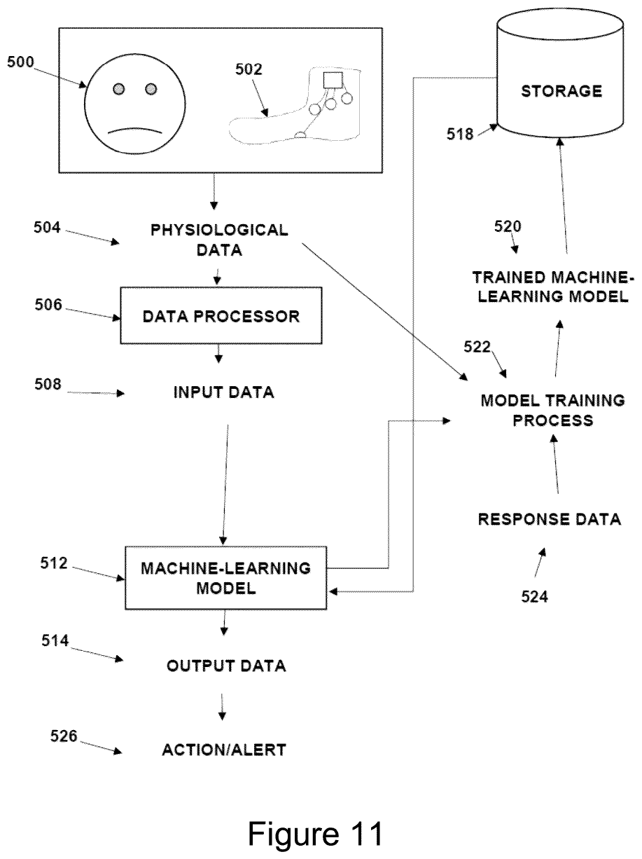

Referring now to FIG. 11, this shows another example system for detecting whether a person 500 is exhibiting a heightened physiological response, in particular stress. A wearable device 502 is worn by person 500; this may be, but need not be, a wearable stress-detection device as described above with reference to FIG. 6.

One or more physiological parameters of the person are measured using one or more sensors of the wearable device 502 worn by the person. For example, the one or more sensors of the wearable device 502 may measure any one of the one or more of the physiological parameters described herein. In an example, the one or more physiological parameters are measured over a period of time, such as 30, 60, or 90 seconds. The resulting measurements may measure the one or more physiological parameters at each time interval during the period of time. For example, a physiological parameter may be measured every 1/10s, 1 s, 10 s, 60 s, etc. Physiological data 504 for the one or more physiological parameters is generated by measuring the one or more physiological parameters as described. For example, the data may be recorded in digital form based upon measurements taken from the one or more sensors.

In some examples, the physiological data 504 may be generated by measuring physiological parameters which were measured during a period of time which overlaps with another period of time for which other physiological data 504 was generated.

The physiological data 504 is then processed by a data processor 506. The data processor 506 may receive the physiological data 504 and determine, based upon the physiological data 504 for each respective physiological parameter, one or more probability values indicative that a physiological parameter within a first range corresponding to a first physiological state will transition to a second range corresponding to a second physiological state. The data processor 506 generates input data 508 based upon the one or more probability values. The data processor 506 may be any suitable processor capable of processing the physiological data 504 to determine the one or more probability values. An example of generating input data based upon probability values is described later.

The input data 508 may be provided as input to a machine-learning model 512. The machine-learning model 512 may be any suitable machine-learning model, such as a convolutional neural network, although, other machine-learning models are envisaged.

Although shown as separate in FIG. 5 the machine-learning model 512 may be implemented on data processor 506. The machine-learning model is configured to receive as input the input data 508 and to generate output data 514 indicating whether the person 500 is exhibiting a heightened physiological response. When implemented as a neural network the machine-learning 512 model may comprise any number or configuration of suitable neural network layers, such as one or more convolutional layers, one or more dense layers, one or more batch normalization layers, one or more dropout layers, and one or more pooling layers. The output data 514 indicating whether the person 500 is exhibiting a heightened physiological response may comprise a binary classification, probability distribution, or any other suitable data. The binary classification may be a classification into one of two classes. For example, the data 514 may be “0” indicating that the person is not exhibiting a heightened physiological response, whereas “1” may indicate otherwise. The probability distribution may include one or more class probability values, such as “0.1” corresponding to class “LOW”, “0.3” corresponding to class “MODERATE”, and “0.6” corresponding to class “HIGH”. In this example, the class probability value with the highest probability corresponds to class “HIGH”, which may indicate that the person is exhibiting a heightened physiological response. In this example, the probability distribution may be {0.1, 0.3, 0.6}. The class with the highest probability may be selected as the stress prediction.

A recommended action or alert 526 may be provided based upon the data indicating whether the person is exhibiting a heightened physiological response. The recommended action 526 may be any of the recommended actions discussed herein. For example, the recommended action 526 may be a recommended action to a carer or observer of the person 500.

In cases where the output data 514 is data indicative that the person is exhibiting a heightened physiological response, a determination as to whether the machine-learning model 512 has previously output data indicating that the person 500 is exhibiting a heightened physiological response in excess of a threshold number of times within a period of time may be made, e.g. 100 (depending on a frequency with which the output is generated). For example, when data is output indicating that the person 500 is exhibiting the heightened physiological response, a sensitivity adjustment process may occur. The sensitivity adjustment process may determine that data indicating that the person 500 is exhibiting a heightened physiological response was previously outputted a particular number of times over the course of 4 minutes. If the threshold number of times is met or exceed and within the period of time, the sensitivity adjustment process may determine that the recommended action 526 is warranted on the basis that both conditions are met. It will be understood that the threshold number of times and the period of time may vary according to whatever values are deemed suitable.

In some examples, the machine-learning model 512 may be trained using a model training process 522. The product of training is a trained machine-learning model 520. The model training process 522 may generate the trained machine-learning model 520 using the machine-learning model 512, the physiological data 504, and response data 524. The response data 524 may be data from the person 500 themselves or from an observer of the person indicating whether the person is exhibiting the heightened physiological response. For example the response data 524 may be received from one or more observers of the person 500, such as a carer. The model training process 522 may use any suitable process to train the machine-learning model 512, such as backpropagation. For example, the response data 524 may be act as ground truth data and the model training process 522 may function to minimize a loss generated by providing the input data 508 as input to the machine-learning model 512. Further detail regarding the model training process 522 is provided with reference to FIG. 9 below. Once the trained machine-learning model 520 is generated, it may be stored for later use. The machine-learning model may be retrained periodically.

In some examples, the machine-learning model 512 may have been pre-trained using a dataset comprising training physiological data for the one or more physiological parameters previously described and ground truth data indicating, for that physiological data, whether one or more individuals other than the person 500 are each exhibiting a heightened physiological response. In other words, physiological parameters of each individual in a population may be measured in order to generate the training physiological data. Corresponding data indicating whether the one or more individuals are each exhibiting the heightened physiological response may be provided. For example, training physiological data may be [0.1, 0.9], [0.7, 0.3]] and [[0.2, 0.8], [0.5. 0.5]] for a first and second individual in a population respectively. In this example, ground truth data may comprise “1” and “0” and may correspond to the training physiological data to indicate that the first individual is “STRESSED” and the second individual is “NOT STRESSED”.

In some examples, prior to providing the input data 508 as input to the machine-learning model, a sensitivity parameter indicating the sensitivity of the person 500 to the heightened physiological response may be received. The sensitivity parameter may be received from a user. For example, a carer of the person 500 may observe the person 500 and indicate that the person 500 is highly sensitivity to the heightened physiological response. The sensitivity parameter may be received in any suitable way, such as by the smartphone or tablet as described herein. In an example, the sensitivity parameter may be received from multiple carers. The sensitivity parameter may be used to generate the input data 508 provided as input to the machine-learning model 512. Further details of the sensitivity parameter are provided below.

One example technique for generating input data based upon one or more probability values is now described. A physiological parameter, e.g. any of the physiological parameters discussed herein with time and transitions between, for illustration, physiological states A, B, C, and D. Physiological states A, B, C, and D may be defined as each having a corresponding (non-overlapping) range of values for the physiological parameter. For example, physiological state A may correspond to a first range A and physiological state D may correspond to a fourth range D.

A probability that a physiological parameter will transition from a first physiological state (e.g. state A) to a second physiological state (e.g. state B) may be modelled using the physiological data described above. In more detail, for a given physiological parameter, a probability that the physiological parameter within a first range for that physiological parameter will transition to a second range for that physiological parameter may be determined using the physiological data for that physiological parameter. One or more probabilities for every combination of each physiological state may be formatted as an adjacency matrix, such as a Markov Transition Matrix. In this way, the probability of each possible transition may be represented. For example, a probability that the physiological parameter will transition from physiological state B to physiological state C may be determined and represented as a first probability value in the Markov Transition Matrix, and so on for B to D, C to A, etc. The one or more probability values may be used to generate input data, in particular input data for input to a machine-learning model as described herein.

In some examples, generating the input data may comprise generating a multi-dimensional array representing the one or more probabilities values for a given physiological parameter. In this example, generating the input data may further comprise combining each multi-dimensional array for one or more physiological parameters to generate an image represented by the input data. Thus, if the physiological parameters were heart rate and electrodermal activity, generating the input data for these physiological parameters may include generating a first multi-dimensional array [0.1, 0.9], [0.2, 0.8] for heart rate and a second multi-dimensional array [[0.3, 0.7], [0.6, 0.4]] for electrodermal activity, and further include combining the first and second multi-dimensional arrays by concatenation, averaging, or any other suitable method of combination. In the example that the physiological parameters were heart rate and electrodermal activity, the input data may be generated as described above using physiological data comprising heart rate data {65 bpm, 85 bpm, 78 bpm, 92 bpm, 115 bpm, 130 bpm, . . . } and electrodermal activity data {0.03 microsiemens, 0.06 microsiemens, 0.12 microsiemens, 0.18 microsiemens, 0.08 microsiemens, 0.12 microsiemens, . . . }.

Any other suitable format for the one or more probabilities is envisaged. For example, the one or more probabilities may be formatted as a unidimensional array (e.g. [0.1, 0.9, 0.2, 0.8]). In the example that the one or more probabilities are formatted as a multi-dimensional array, the multi-dimensional array may be a matrix.

In an example, a probability value Pij quantifying the probability of transition between state Si to state Sj is calculated as a ratio between a number of transitions between state Si and Sj in a set of time series observations (e.g. the physiological data for a given physiological parameter) and the total number of transitions from state Si to any other possible state. It will be understood that the one or more probabilities may be determined in any other suitable way.

A Markov Transition Field can be an image generated based upon the one or more probability values, e.g. as previously described. The image may be input data to be provided as input to the machine-learning model as described herein. The image may comprise one or more channels each corresponding to a different physiological parameter. For example, a first channel of the image may represent one physiological parameter, whereas a second and a third channel may each represent another respective physiological parameter (analogous with red, green, and blue colour image channels). In this way, the image can capture temporal dynamics of the one or more physiological parameters. Also the Markov Transition Field efficiently compresses information regarding the physiological parameters conveniently for further processing, e.g. by the machine-learning model 512 of FIG. 5. In implementations the Markov Transition Field comprises a multichannel image with dimensions (10, 10, 9), i.e. 9 different channels (for 9 different physiological parameters) each including 100 probability values for transitions between every possible pair of 10 physiological states.

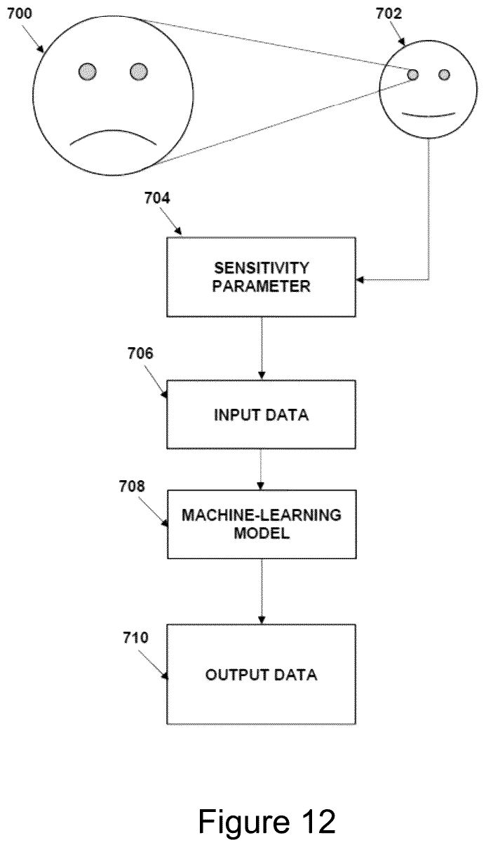

FIG. 12 schematically depicts a system user 702, in this example observing a person 700, a sensitivity parameter 704, input data 706, a machine-learning model 708, and output data 708. The sensitivity parameter 704 generally provides a means for sensitivity adjustment, i.e. an ability of the machine-learning model to be adjusted to a person's 700 sensitivity to the heightened physiological response. If the person 700 is highly sensitivity to the heightened physiological response, the machine-learning model 700 may generate output data 708 that causes recommended actions to be provided to individuals, such as the person's 700 carer, at an overly fast rate. In such cases, sensitivity adjustment may be employed in order to modulate output from the machine-learning model. Sensitivity adjustment also provides a means for improving the accuracy of predictions as to whether the person 700 is genuinely exhibiting a (biomarker of a) heightened physiological response.

In FIG. 12, the sensitivity parameter 704 may be received from system user 702. The sensitivity parameter 704 may be a value indicating the sensitivity of the person 700 to the heightened physiological response.

In some implementations the sensitivity parameter sets a threshold between the output classes “no stress” and “stress”. If the sensitivity is high, lower levels of stress present in the physiological data are classified as “stress”. If the sensitivity is low, higher levels of stress must be present in the physiological data to generate a “stress” output from the classifier. The sensitivity has a default setting (normal), but can be adjusted for each individual wearer (high/low), e.g. by the carer. In these implementations the sensitivity adjustment mechanism can sit outside of a learning/adaptation mechanism that uses (carers') observations of distress/behaviour to re-train or fine tune the classifier.

The sensitivity parameter 704 may be received from a plurality of users, for example, by aggregating the values given by each one of the users. The sensitivity parameter 704 may be received from the one or more users through a device, such as a smartphone or tablet. In some examples, a web application enables the user(s) to input a sensitivity parameter for a given person 700.

The sensitivity parameter 704 may be used to generate the input data 706. For example, the input data 706 may be generated based upon probability values [0.3, 0.7], [0.6, 0.4] and sensitivity parameter 704 “0.8”. In this example, the input data may comprise data representing the probability values and data representing the sensitivity parameter 704. In this example, providing the input data 706 as input to the machine-learning model 708 to generate output data 710 may comprise providing the data representing the sensitivity parameter as input to a final layer of the machine-learning model 708, and optionally comprise providing the data representing the probability values as input to a first layer of the machine-learning model 708. It will be understood that any suitable arrangement for providing the input data 706 as input to the machine-learning model 708 is envisaged.

In response to providing the input data 706 as input to the machine-learning model 708, the machine-learning model 708 may generate the output data 710. The output data 710 may be characterized as being sensitivity adjusted in the case that the input data 706 was generated based upon the sensitivity parameter 704.

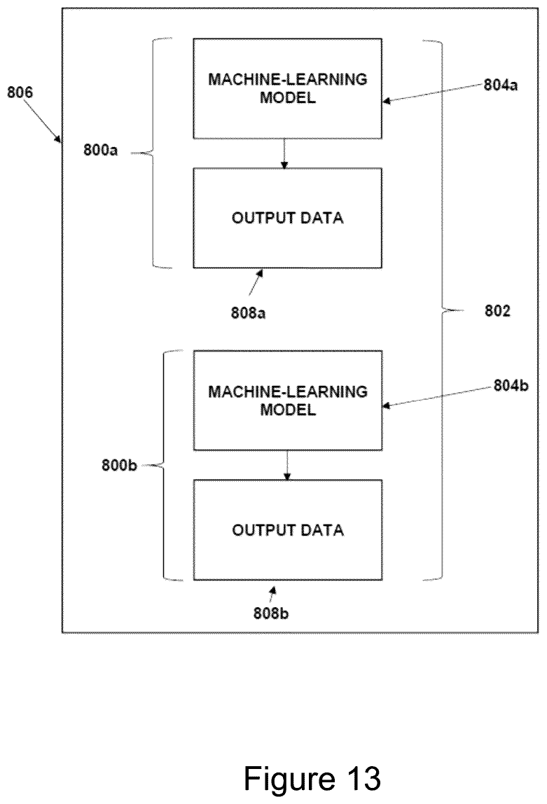

FIG. 13 schematically illustrates a sensitivity adjustment process 806 for sensitivity adjustment, thereby providing a further means for sensitivity adjustment over those described with reference to FIG. 12. The sensitivity adjustment process 806 may be the sensitivity adjustment process described with reference to FIG. 11.

In FIG. 13, a machine-learning model 804a generates output data 808a at a first point in time 800a, and the same machine-learning model 804b generates output data 808b at a second point in time 800b. In FIG. 13, it should be understood that the same machine-learning model 804 may generate multiple different output data during a period of time 802. If the machine-learning model 804b generates output data 808b that is indicative that a person is exhibiting the heightened physiological response, the sensitivity adjustment process may determine whether the machine-learning model 804b has previously outputted data indicating that the person is exhibiting a heightened physiological response, for example, at the first point in time 800a. The sensitivity adjustment process may further determine whether the machine-learning model 804b has done so in excess of a threshold number of times and/or within a period of time. For example, the sensitivity adjustment process may determine that the machine-learning model 804b has generated output data 808b a particular number of times previously, such as output data 808a. Further, the sensitivity adjustment process may also determine that the machine-learning model 804b has generated output data 808b within the period of time 802, such as within a period of 3 to 5 minutes. In response, the sensitivity adjustment process can determine not to take further action until the suitable conditions are met as described are met. For example, the sensitivity adjustment process can determine not to provide recommended actions until the machine-learning model 804 has generated output data 808 indicative that the person is exhibiting the heightened physiological response more than a threshold number of times within 3 to 5 minutes.

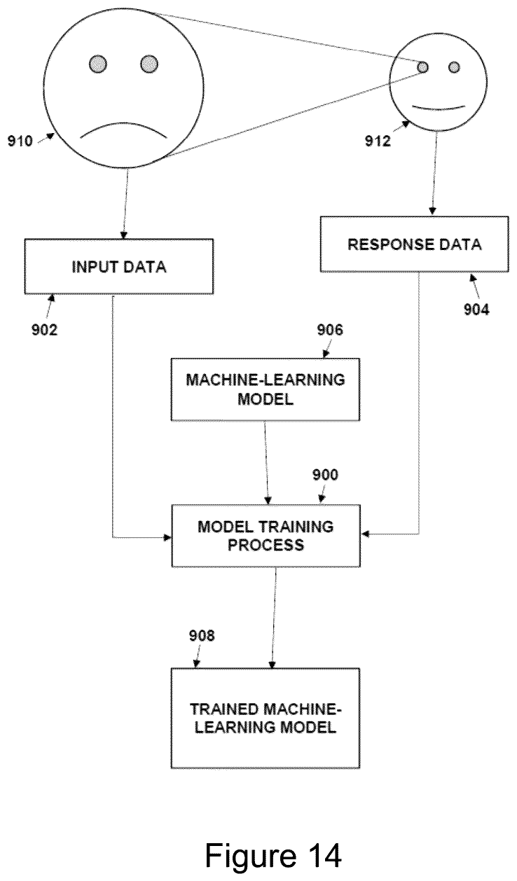

FIG. 14 schematically illustrates a model training process 900. The model training process 900 comprises using input data 902, response data 904, and a machine-learning model 906 in order to generate a trained machine-learning model 908. FIG. 14 further depicts an observer 912 observing a person 910. The observer 912 and the person 910 may be the observer and person described previously. The model training process 900 provides a means for initializing the machine-learning model 906 such that the machine-learning model 906 is adapted and specific to the person 910. The model training process 900 further provides a means for updating the machine-learning model 906 in the event that further data is required to properly detect whether the person 910 is exhibiting the heightened physiological response.

The model training process 900 may receive the response data 904 from the observer 912 of the person 910. The response data 904 may indicate whether the person 910 is exhibiting the heightened physiological response. The response data 904 may be received by the model training process 900 in any suitable way. For example, the observer 912 may input an observation, i.e. that the person 910 is exhibiting (a biomarker of) the heightened physiological response, to a smartphone or tablet and the model training process 900 may receive the response data 904 therefrom. The model training process 900 may further receive the input data 902. The input data 902 may be as previously described. The input data 902 may be obtained by generating physiological data measuring physiological parameters of the person 910, and subsequently generating the input data 902 using probability values determined based upon the physiological data. Hence, the input data 902 may correspond to the response data 904. The response data 904 may act as ground truth data. In other words, the response data 904 may indicate whether the person is exhibiting the heightened physiological response, based on the observations of one or more observers of the person, thereby providing authoritative and accurate information regarding whether the input data 902 is indicative of the person 910 exhibiting the heightened physiological response.

The model training process 900 may receive the machine-learning model 906 for training, e.g. in response to determining that training is required, e.g. based on whether or not a threshold period of time has elapsed or based on a performance evaluation of the model, e.g. by using test (training) data and evaluating a value of the training objective (loss) function.

The machine-learning model 900 received for training may be a machine-learning model that has been trained on a dataset for a population of individuals other than the person 910 as described above. The model training process 900 may use the input data 902 and the response data 904 to train the machine-learning model 906 and generate the trained machine-learning model 908. As described above, the trained machine-learning model 908 may subsequently be stored, and the machine-learning model 906 may be a previously trained machine-learning model 908 retrieved from storage. Storing and retrieving the trained machine-learning model 908 and machine-learning model 906 respectively may be achieved using any suitable means, such as over a network, e.g. using the transmitting means described herein.

The model training process 900 may perform any suitable method of training the machine-learning model 906, such as backpropagation with gradient descent. For example, the machine-learning model 906 may be iteratively updated by providing the input data 902 as input to the machine-learning model 906, calculating a loss, e.g. a difference between an output of the machine-learning model 906 based on the input data 902 and the response data 904. This can involve providing the input data 902 as input to the machine-learning model 906, calculating gradients for the machine-learning model's 906 parameters indicating how the parameters should be adjusted, and updating the machine-learning model's 906 parameters based upon those calculated gradients.

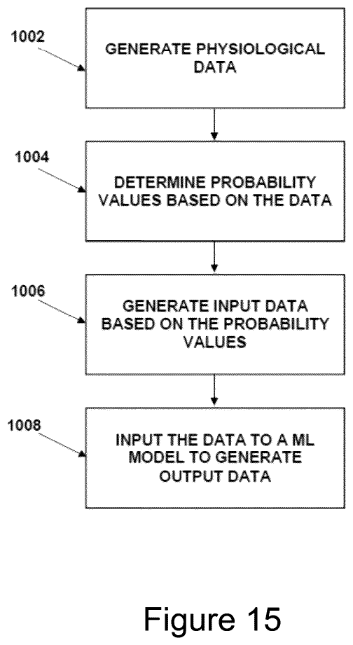

FIG. 15 is a flow diagram of a method of detecting whether a person is exhibiting a heightened physiological response. The method may be carried out as previously described. In some examples, the heightened physiological response is physiological stress.

At step 1002, physiological data for one or more physiological parameters is generated by measuring one or more physiological parameters of a person using one or more sensors of a wearable device worn by the person. In some examples, the one or more physiological parameters are each one of heart-rate, heart rate variability, linear interpolation of heart rate, electrodermal activity (EDA), EDA skin conductance response, EDA skin conductance level, skin temperature or an activity index. In some examples, the wearable device is in the form of a sock. In some examples, the one or more physiological parameters are measured during a first period of time. In some examples, the method further comprises measuring the one or more physiological parameters of the person using the one or more sensors of the wearable device worn by the person during a second period of time, the second period of time overlapping with the first period of time. In some examples, the first and second period of time are 30, 60 or 90 seconds.

At step 1004, one or more probability values indicative that a physiological parameter within a first range corresponding to a first physiological state will transition to a second range corresponding to a second physiological state are determined based upon the physiological data. In some examples, the first physiological state is indicative that the person is not exhibiting a heightened physiological response. In some examples, the second physiological state is indicative that the person is exhibiting a heightened physiological response.

At step 1006, input data is generated based upon the one or more probability values. In some examples, generating the input data comprises generating, for each of the one or more physiological parameters, a multi-dimensional array representing the respective one or more probability values for the physiological parameter. In some examples, generating the input data further comprises combining each multi-dimensional array in order to generate an image represented by the input data. In some examples, the multi-dimensional array is a matrix. In some examples, the image is a Markov Transition Field. In some examples, the image comprises one or more channels each corresponding to a different one of the one or more physiological parameters. In some examples, combining each multi-dimensional array comprises concatenating the one or more multi-dimensional arrays, or averaging the one or more multi-dimensional arrays. In some examples, the image comprises one or more channels each corresponding to a different one of the one or more physiological parameters. In some examples, the method further comprises receiving, from one or more observers of the person, a sensitivity parameter indicating the sensitivity of the person to the heightened physiological response, where the generating the input data is further based upon the sensitivity parameter.

At step 1008, input data is provided as input to a machine-learning model to generate output data indicating whether the person is exhibiting a heightened physiological response. In some examples, the machine-learning model comprises a convolutional neural network. In some examples, the output data comprises a binary classification indicating whether or not the person is exhibiting the heightened physiological response. In some examples, the output data comprises a probability distribution comprising one or more class probability values, each class probability value indicating a probability that the person is exhibiting a different level of heightened physiological response. In some examples, the method further comprises determining whether the machine-learning model has previously generated output data indicating that the person is exhibiting a heightened physiological response in excess of a threshold number of times within a period of time, where the output data is indicative that the person is exhibiting a heightened physiological response. In some examples, the period of time is 3 to 5 minutes. In some examples, the threshold number of times is 100. In some examples, the machine-learning model comprises one or more convolutional layers, one or more dense layers, one or more batch normalization layers, one or more dropout layers, and one or more pooling layers. In some examples, the machine-learning model was pre-trained using a dataset comprising training physiological data for the one or more physiological parameters, the training physiological data generated by measuring the one or more physiological parameters of one or more individuals other than the person and ground truth data indicating, for the physiological data, whether the one or more individuals other than the person are each exhibiting a heightened physiological response. In some examples, the method further comprises providing, based upon the output data, a recommended action. In some examples, the method further comprises receiving, from one or more observers of the person, response data indicating whether the person is exhibiting the heightened physiological response, generating a trained machine-learning model by training the machine-learning model using the input data and the response data, and storing the trained machine-learning model. In some examples, the method further comprises retrieving the trained machine-learning model from storage, and in response to determining that a threshold period of time has lapsed, determining that further training of the machine-learning model is not required.

Further aspects are defined in the following clauses:

1. A method of detecting a stress condition of a person, the method comprising the steps of: measuring one or more physiological parameters of the person to generate an input variable for each of the one or more physiological parameters, using a stress type classifier to evaluate the one or more input variables to determine a type of stress being experienced by the person, selected from a plurality of pre-determined types of stress, and generating a stress type output signal corresponding to the determined stress type.
2. A method of detecting a stress condition of a person according to clause 1, wherein the plurality of pre-determined types of stress comprises one or more of a pain anxiety, or generalised non-specific stress.
3. A method of detecting a stress condition of a person according to clauses 1 or 2, wherein the one or more physiological parameters comprises one or more or heart rate, heart rate variability, linear interpolation of heart rate, electrodermal activity, electrodermal activity skin conductance level, electrodermal activity skin conductance response or skin temperature.
4. A method of detecting a stress condition of a person according to any of clauses 1-3, wherein the stress type output signal includes a probabilistic score.
5. A method of detecting a stress condition of a person according to clauses 1-4, further comprising a stress intensity classifier to determine a stress state of the person corresponding to the intensity of the stress experienced by the person and generating a stress intensity output signal corresponding to the determined stress state.
6. A method of detecting a stress condition of a person according to clause 5, wherein the stress intensity output signal includes a probabilistic score of the stress state of the user.
7. A method of detecting a stress condition of a person according to any of the previous clauses, wherein the stress type classifier is trained on pre-existing data corresponding to each of the plurality of pre-determined types of stress for each of the one or more physiological parameters.
8. A method of detecting a stress condition of a person according to any preceding clause, where a measure of electrodermal activity skin conductance level is one of the physiological parameters, and at least one of the mode, median or mean of the anxiety probability density function is greater than the corresponding mode, median or mean of both the pain and generalised non-specific stress probability density functions for electrodermal activity skin conductance level.
9. A method of detecting a stress condition of a person according to any preceding clause, where a measure of electrodermal activity skin conductance response is one or the physiological parameters, and at least one of the mode, median or mean of the anxiety probability density function is greater than the corresponding mode, median or mean of both the pain and generalised non-specific stress probability density functions for electrodermal activity skin conductance response.
10. A method of detecting a stress condition of a person according to any preceding clause, where a measure of heart rate is one of the one or more physiological parameters, and at least one of the mode, median or mean of the anxiety probability density function is greater than the corresponding mode, median or mean of both the pain and generalised non-specific stress probability density functions for heart rate.
11. A method of detecting a stress condition of a person according to any preceding clause, where a measure of heart rate inter-beat interval is one of the one or more physiological parameters, and at least one of the mode, median or mean of the anxiety probability density function is greater than the corresponding mode, median or mean of both the pain and generalised non-specific stress probability density functions for heart rate inter-beat interval.
12. A method of detecting a stress condition of a person according to any preceding clause, where linear interpolation of heart rate is one of the one or more physiological parameters, and at least one of the mode, median or mean of the anxiety probability density function is greater than the corresponding mode, median or mean of both the pain and generalised non-specific stress probability density functions for linear interpolation heart rate.
13. A method of detecting a stress condition of a person according to any preceding clause, where skin temperature is one of the one or more physiological parameters, and at least one of the mode, median or mean of the anxiety probability density function is less than the corresponding mode, median or mean of both the pain and generalised non-specific stress probability density functions for skin temperature.
14. A method of detecting a stress condition of a person according to any preceding clause, where the classifier determined probability scores for each stress type, which are compared to determine the most likely type of stress being experienced by the user, selected from the plurality of pre-determined types of stress.
15. A method of detecting a stress condition of a person according to any of the previous clauses, wherein the stress type output signal is transmitted to an electronic device.
16. A method of detecting a stress condition of a person according to any of the previous clauses, where an accelerometer is used to determine the repetitive motion of the user.
17. A method of detecting a stress condition of a person according to any preceding clause, where the stress intensity classifier comprises a person's individual dataset containing baseline data of the user under a stressed and/or non-stressed state.
18. A method of detecting a stress condition of a person according to any of the previous clauses, where one or more recommended actions based on the stress type output signal are provided as an output.
19. A device for detecting a stress condition of a person, comprising: one or more sensors for measuring one or more physiological parameters, and a controller configured to perform the method for detecting a stress condition of a person as recited in any preceding clause.
20. A device for detecting a stress condition of a person according to clause 19, wherein the stress-detection device comprises a wearable garment, and one or more sensors are at least partially integrated into the wearable garment.
21. A device for detecting a stress condition of a person according to clause 20, wherein the wearable garment is a sock.
22. A computer-implemented method of detecting whether a person is exhibiting a heightened physiological response, the method comprising: generating, by measuring one or more physiological or motion parameters of the person using one or more sensors of a wearable device worn by the person, sensor data for the one or more physiological or motion parameters; determining, based upon the sensor data for each respective parameter, one or more probability values indicative that a parameter within a first range corresponding to a first physiological or motion state will transition to a second range corresponding to a second physiological or motion state; generating input data based upon the one or more probability values; and providing the input data as input to a machine-learning model to generate output data indicating whether the person is exhibiting a heightened physiological response; in particular wherein the wearable device is in the form of a sock; and optionally providing, based upon the output data, a recommended action or alert.
23. The computer-implemented method of clause 22, wherein, prior to training the machine-learning model, the machine-learning model was pre-trained using a dataset comprising: training sensor data for the one or more physiological parameters, the training sensor data generated by measuring the one or more physiological or motion parameters of one or more other people; and ground truth data indicating, for the sensor data, whether the one or more other people are each exhibiting a heightened physiological response.
24. The computer-implemented method of clause 22 or 23, wherein the one or more physiological or motion parameters are measured during a first period of time; and further comprising: measuring the one or more physiological or motion parameters of the person using the one or more sensors of the wearable device worn by the person during a second period of time, the second period of time overlapping with the first period of time; and generating the output data for each of the first and second periods of time.
25. A wearable device for detecting whether a person is exhibiting a heightened physiological response, comprising: one or more sensors for measuring one or more physiological parameters; and a controller configured to perform a method according to any one of clauses 22-24.
26. A method of monitoring a person, comprising: obtaining time series sensor data from one or more sensors of the person, the time series sensor data comprising a series of one or both of physiological data from one or more physiological sensors characterizing a physiological state of the person and motion data from one or more motion sensors characterizing motion of the person; obtaining a sequence of observations of the time series sensor data; processing the sequence of observations to generate a representation of the sequence of observations as a two-dimensional image that represents relationships between the observations in the sequence; and processing the two-dimensional image using a neural network comprising one or more convolutional neural network layers to generate classification data that predicts, for the sequence of observations, one of a plurality of categories, wherein the categories characterize different respective predicted motion states of the person.
27. The method of clause 26, comprising obtaining the time series sensor data from one or more sensors of a sock worn by the person.

No doubt many other effective alternatives will occur to the skilled person. It will be understood that the invention is not limited to the described embodiments and encompasses modifications apparent to those skilled in the art lying within the spirit and scope of the claims appended hereto.

1. A method of monitoring a person, comprising:
obtaining time series sensor data from one or more sensors of a sock worn by the person, the time series sensor data comprising one or both of i) physiological data from one or more physiological sensors characterizing a physiological state of the person and ii) motion data from one or more motion sensors characterizing motion of the person;
obtaining a sequence of observations of the time series sensor data; and
processing the sequence of observations using a machine learning classifier to generate classification data that predicts, for the sequence of observations, one of a plurality of categories, wherein the categories characterize different respective predicted states of stress of the person.

2. The method of claim 1, wherein the time series sensor data comprises a series of physiological data from one or more physiological sensors, and wherein the sock is used to bring one or more of the physiological sensors into direct contact with the skin of the person to collect the physiological data.

3. The method of claim 1, wherein the one or more sensors comprise a sensor to sense electrodermal activity of the skin of the person and an accelerometer.

4. The method of claim 1, further comprising providing an alert in response to the generated classification data indicating a change in the predicted state of stress.

5. The method of claim 1, further comprising treating the person to reduce stress in response to the generated classification data indicating that the person has a heightened predicted state of stress.

6. The method of claim 1, wherein obtaining the sequence of observation comprises applying a rolling window to the time series sensor data, wherein the rolling window has a duration of at least 30 seconds.

7. The method of claim 1, wherein processing the sequence of observations using the machine learning classifier comprises:
processing the sequence of observations to generate a representation of the sequence of observations as a two-dimensional image that represents relationships between the observations in the sequence; and
processing the two-dimensional image using a neural network comprising one or more convolutional neural network layers to generate the classification data; and
wherein the classification data comprises a score for each category of the plurality of categories, each score representing an estimated likelihood of a respective one of the states of stress.

8. The method of claim 7, wherein processing the sequence of observations to generate the representation of the sequence of observations as the two-dimensional image comprises generating a Markov Transition Field or Gramian Angular Field representation of the sequence of observations.

9. The method of claim 1, wherein processing the sequence of observations using the machine learning classifier comprises:
processing the sequence of observations to generate a set of features or metrics representing the sequence of observations, wherein the metrics comprise one or more of mean, median, standard deviation, and interquartile range; and
processing the set of features or metrics using a decision tree model to generate the classification data.

10. The method of claim 1, wherein the machine learning classifier comprises a neural network having a plurality of neural layers and an output layer configured to apply a softmax function, and wherein processing the sequence of observations using the machine learning classifier comprises:
processing the sequence of observations using a neural network to generate classification data comprising a score for each category of the plurality of categories, each score representing an estimated likelihood of a respective one of the states of stress;
obtaining a control input that specifies a sensitivity of the monitoring; and
adjusting a temperature parameter of the softmax function using the control input to adjust the sensitivity of the monitoring.

11. The method of claim 1, comprising:
obtaining training data from a particular person; and
fine-tuning the machine learning classifier using the training data; and
using the fine-tuned machine learning classifier to monitor the particular person.

12. A computer-implemented method of detecting whether a person is exhibiting a heightened physiological response, the method comprising:
generating, by measuring one or more physiological or motion parameters of the person using one or more sensors of a wearable device worn by the person, sensor data for the one or more physiological or motion parameters;
determining, based upon the sensor data for each respective parameter, one or more probability values indicative that a parameter within a first range corresponding to a first physiological or motion state will transition to a second range corresponding to a second physiological or motion state;
generating input data based upon the one or more probability values; and
providing the input data as input to a machine-learning model to generate output data indicating whether the person is exhibiting a heightened physiological response;
wherein the wearable device is in the form of a sock; and
providing, based upon the output data, a recommended action or alert.

13. The computer-implemented method of claim 12, wherein the one or more physiological or motion parameters are each one of heart-rate, heart rate variability, linear interpolation of heart rate, electrodermal activity (EDA), EDA skin conductance response, EDA skin conductance level, skin temperature or an activity index; wherein the first physiological state is indicative that the person is not exhibiting a heightened physiological response, and wherein the second physiological state is indicative that the person is exhibiting a heightened physiological response.

14. The computer-implemented method of claim 12, wherein the machine-learning model is a convolutional neural network, wherein the machine-learning model comprises one or more convolutional layers, one or more dense layers, one or more batch normalization layers, one or more dropout layers, and one or more pooling layers; wherein the output data comprises a binary classification indicating whether or not the person is exhibiting the heightened physiological response, or a probability distribution comprising one or more class probability values, each class probability value indicating a probability that the person is exhibiting a different level of heightened physiological response.

15. The computer-implemented method of claim 12, the method further comprising determining whether the machine-learning model has previously generated output data indicating that the person is exhibiting a heightened physiological response in excess of a threshold number of times within a period of time, and wherein the output data is indicative that the person is exhibiting a heightened physiological response; wherein the period of time is 3 to 5 minutes.

16. The computer-implemented method of claim 12, further comprising:
receiving, from a user, a sensitivity parameter indicating the sensitivity of the person to the heightened physiological response; and
wherein generating the input data is further based upon the sensitivity parameter.

17. The computer-implemented method of claim 12, wherein generating the input data comprises:
generating, for each of the one or more physiological parameters, a multi-dimensional array representing the respective one or more probability values for the physiological parameter; and
combining, by concatenating or averaging, each multi-dimensional array in order to generate an image represented by the input data;
wherein the multi-dimensional array is a matrix, wherein the image is a Markov Transition Field, wherein the image comprises one or more channels each corresponding to a different one of the one or more physiological parameters.

18. The computer-implemented method of claim 12, further comprising:
receiving, from the person or one or more observers of the person, response data indicating whether the person is exhibiting the heightened physiological response;
generating a trained machine-learning model by training the machine-learning model using the physiological data and the response data; and
storing the trained machine-learning model; further comprising:
retraining machine-learning model periodically.

19. One or more non-volatile data carriers carrying processor control code that when executed by one or more processors cause the one or more processors to implement the method of claim 1.

20. A system for monitoring a person, comprising:
a sock to be worn by a person, the sock comprising one or both of i) one or more physiological sensors to obtain physiological data characterizing a physiological state of the person and ii) motion sensors to obtain motion data characterizing motion of the person; and
one or more computers storing processor control code that, when executed by one or more processors of the one or more computers, cause the one or more computers to implement the method of claim 1.
